SEQUENCE LISTING (9 sequences)

SEQ ID NO: 1 - DNA, 32 bp, Artificial Sequence
  Synthetic
aaaaaaaaaatgtgaatagacttgccatacgt

SEQ ID NO: 2 - DNA, 26 bp, Artificial Sequence
  Synthetic
aaaaaaaaaaacgtatggcaagtcta

SEQ ID NO: 3 - DNA, 22 bp, Artificial Sequence
  Synthetic
acgtatggcaagtctattcaca

SEQ ID NO: 4 - DNA, 32 bp, Artificial Sequence
  Synthetic
aaaaaaaaaataccgtaattcttgagaccagg

SEQ ID NO: 5 - DNA, 26 bp, Artificial Sequence
  Synthetic
aaaaaaaaaacctggtctcaagaatt

SEQ ID NO: 6 - DNA, 22 bp, Artificial Sequence
  Synthetic
cctggtctcaagaattacggta

SEQ ID NO: 7 - DNA, 32 bp, Artificial Sequence
  Synthetic
aaaaaaaaaagcattgttccctagcgtcatct

SEQ ID NO: 8 - DNA, 25 bp, Artificial Sequence
  Synthetic
aaaaaaaaaaagatgacgctaggga

SEQ ID NO: 9 - DNA, 22 bp, Artificial Sequence
  Synthetic
agatgacgctagggaacaatgc

(19) United States
(12) Patent Application Publication
(10) Pub. No.: US 2014/0100131 A1
(43) Pub. Date: Apr. 10, 2014

(54) Methods and Reagents for Target Isolation from a Sample

(71) Applicants: Xiaohu GAO, Seattle, WA (US); Christine PROBST, Seattle, WA (US); Pavel S. ZRAZHEVSKIY, Redmond, WA (US)

(72) Inventors: Xiaohu GAO, Seattle, WA (US); Christine PROBST, Seattle, WA (US); Pavel S. ZRAZHEVSKIY, Redmond, WA (US)

(21) Appl. No.: 14/050,702

(22) Filed: Oct. 10, 2013

Related U.S. Application Data
(60) Provisional application No. 61/712,102, filed on Oct. 10, 2012.

(51) Int. Cl. G01N 33/543

(52) U.S. Cl. CPC G01N 33/54353
USPC 506/9; 436/501; 435/5; 435/6.11; 506/16

(57) ABSTRACT

The present invention provides methods and kits for isolating a target of interest from a sample, where the methods involve use of nucleic acid strand displacement technology.

### CROSS REFERENCE

This application claims priority to U.S. Provisional Patent Application Ser. No. 61/712,102, filed Oct. 10, 2012, incorporated by reference herein in its entirety.

### STATEMENT OF GOVERNMENT SUPPORT

This invention was made with government support under grant no. R01 CA131797 awarded by the National Institutes of Health, grant no. 0645080 awarded by the National Science Foundation, and graduate research fellowship no. DGE-0718124 awarded by the National Science Foundation. The government has certain rights in the invention.

### BACKGROUND

Medical and biological research is progressing at an astonishing rate, with an increasing emphasis on the understanding of complex biological systems. Such progress requires the development of technologies that take more biological measurements, with increasing sensitivity and precision, at higher speed and lower cost. Thus, methods and reagents that improve the speed, sensitivity, and/or precision of biological target isolation are needed in the art.

### SUMMARY OF THE INVENTION

In a first aspect, the present invention provides methods for target isolation, comprising
(a) generating a first complex in a sample, wherein the first complex comprises:
   (i) a first single stranded nucleic acid (NA1);
   (ii) a second single stranded nucleic acid (NA2) bound to a surface, wherein the NA2 is complementary to the NA1, wherein the NA2 is bound to the NA1 via base pairing to form an NA1-NA2 complex comprising a double stranded region of at least 7 base pairs;
   (iii) a first binding molecule (B1) bound to the NA1; and
   (iv) a first target (T1) from the sample bound to the binding molecule; wherein the first complex comprises a T1-B1-NA1-NA2 complex;
(b) contacting the T1-B1-NA1-NA2 complex with a third single stranded nucleic acid (NA3); wherein the NA3 is complementary to a contiguous portion of either the NA1 or NA2 present in the double stranded region, such that hybridization of the NA3 to either the NA1 or NA2 is thermodynamically more favorable than the NA1-NA2 double stranded region; wherein the contacting occurs for a time and under conditions suitable to promote base pairing between the NA3 and either the NA1 and the NA2 to form a double stranded NA1-NA3 or NA2-NA3 region, whereby the NA1-NA2 double stranded region is disrupted; and
(c) isolating the T1 from the sample.

In one embodiment, generating the T1-B1-NA1-NA2 complex comprises:
(A) contacting the sample of interest with the NA1 bound to the B1; wherein the contacting occurs for a time and under conditions suitable for binding of the B1 to the T1 in the sample to form a T1-B1-NA1 complex;
(B) contacting the NA2 with the surface for a time and under conditions suitable to promote NA2 binding to the surface, thereby forming a surface-bound NA2;
(C) contacting the T1-B1-NA1 complex with the surface-bound NA2; wherein the contacting occurs for a time and under conditions suitable to promote formation of the NA1-NA2 complex, thereby forming the T1-B1-NA1-NA2 complex.

In another embodiment, generating the T1-B1-NA1-NA2 complex comprises:
(A) contacting the NA2 with the surface for a time and under conditions suitable to promote NA2 binding to the surface, thereby forming a surface-bound NA2;
(B) contacting the NA1 bound to the B1 with the surface-bound NA2; wherein the contacting occurs for a time and under conditions suitable to promote formation of the NA1-NA2 complex, thereby forming a B1-NA1-NA2 complex; and
(C) contacting the B1-NA1-NA2 complex with the sample of interest, wherein the contacting occurs for a time and under conditions suitable for binding of the B1 to the T1 in the sample to form the T1-B1-NA1-NA2 complex

In a further embodiment, generating the T1-B1-NA1-NA2 complex comprises:
(A) contacting the sample of interest with the NA1 bound to the B1; wherein the contacting occurs for a time and under conditions suitable for binding of the B1 to the T1 in the sample to form a T1-B1-NA1 complex;
(B) contacting the T1-B1-NA1 complex with the NA2; wherein the contacting occurs for a time and under conditions suitable to promote formation of the NA1-NA2 complex; and
(C) contacting the T1-B1-NA1-NA2 complex with the surface for a time and under conditions suitable to promote NA2 binding to the surface, thereby forming the complex.

In one embodiment, steps (a) and (b) are each carried out two or more times. In another embodiment, the surface comprises a surface selected from the group consisting of magnetic particles, microarrays, beads, columns, optical fibers, wipes, nitrocellulose, nylon, glass, quartz, diazotized membranes, silicones, polyformaldehyde, cellulose, cellulose acetate, paper, ceramics, metals, metalloids, semiconductive materials, coated beads, plastics, gel-forming materials, sol gels, porous polymer hydrogels, nanostructured surfaces, nanotubes, and nanoparticles. In a further embodiment, the NA1-NA2 complex double stranded region is between about 7 base pairs and about 30 base pairs in length. In another embodiment, the NA1-NA2 complex double stranded region has a melting temperature of about 10° C. or above. In a still further embodiment, the double stranded NA1-NA3 or NA2-NA3 region is between about 8 base pairs and about 50 base pairs in length.

In another embodiment, the method further comprises
(d) generating a second complex in the sample, wherein the second complex comprises:
   (i) a first single stranded nucleic acid (NA4);
   (ii) a second single stranded nucleic acid (NA5) bound to a surface, wherein the NA5 is complementary to the NA4, wherein the NA5 is bound to the NA4 via base pairing to form an NA4-NA5 complex comprising a double stranded region of at least 7 base pairs;
   (iii) a second binding molecule (B2) bound to the NA4; and
   (iv) a second target (T2) from a sample of interest bound to the binding molecule; wherein the complex comprises a T2-B2-NA4-NA5 complex;
(e) contacting the T2-B2-NA4-NA5 complex with a third single stranded nucleic acid (NA6); wherein the NA6 is complementary to a contiguous portion of either the NA4 or NA5 present in the double stranded region, such that hybridization of the NA6 to either the NA4 or NA5 is thermodynamically more favorable than the NA4-NA5 double stranded region; wherein the contacting occurs for a time and under conditions suitable to promote base pairing between the NA6 and either the NA4 or the NA5, whereby the NA4-NA5 double stranded region is disrupted; and
(f) isolating the T2 from the sample.

In another embodiment, methods for multiplexed target isolation, comprise
(a) generating a plurality of unique complexes in a sample, wherein each unique complex comprises:
   (i) a unique first single stranded nucleic acid (UNA1);
   (ii) a unique second single stranded nucleic acid (UNA2) bound to a surface, wherein the UNA2 is complementary to the UNA1, wherein the UNA2 is bound to the UNA1 via base pairing to form an UNA1-UNA2 complex comprising a double stranded region of at least 7 base pairs;
   (iii) a unique binding molecule (UB) bound to the UNA1; and
   (iv) a unique target (UT) from a sample of interest bound to the UB; wherein each unique complex comprises a UT-UB-UNA1-UNA2 complex;
(b) sequentially contacting each UT-UB-UNA1-UNA2 complex with a unique third single stranded nucleic acid (UNA3); wherein the UNA3 is complementary to a contiguous portion of either the UNA1 or UNA2 present in the double stranded region such that hybridization of the UNA3 to either the UNA1 or UNA2 is thermodynamically more favorable than the UNA1-UNA2 double stranded region; wherein the contacting occurs for a time and under conditions suitable to promote base pairing between the UNA3 and either the UNA1 or the UNA2, whereby the UNA1-UNA2 double stranded region is disrupted; and
(c) isolating each unique target from the sample.

In another aspect, the invention provides kits, comprising:
(a) a first single stranded nucleic acid (NA1);
(b) a second single stranded nucleic acid (NA2) capable of binding to a surface, wherein the NA2 is complementary to the NA1, wherein the NA2 is capable of binding to the NA1 via base pairing to form an NA1-NA2 complex a double stranded region of at least 7 base pairs; and
(c) a third single stranded nucleic acid (NA3); wherein the NA3 is complementary to a contiguous portion of either the NA1 or NA2 present in the double stranded region such that hybridization of the NA3 to either the NA1 or NA2 is thermodynamically more favorable than the NA1-NA2 double stranded region.

### DESCRIPTION OF THE FIGURES

FIG. 1: Schematic of multi-target immunomagnetic sorting. (a) SMD-based sorting technology captures all targets of interest simultaneously, followed by a rapid sorting through release of MB-Target link. (b) Target is captured through immunorecognition by DNA-encoded antibody and partial hybridization with CP on MB. Selective target release is achieved through sequence-specific EP displacement due to a more favorable hybridization between CP and DP.

FIG. 2: Kinetics and specificity of target capture in single-target experiment. Quantitative analysis is performed for target capture through (i) streptavidin-biotin bond formation and (ii) complementary DNA hybridization in comparison to control cases utilizing (iii) non-complementary CP-EP sequences, (iv) non-targeting antibody, and (v) MB-CP alone. Average of 3 separate experiments is shown. Error bars indicate one standard deviation.

FIG. 3: Kinetics and specificity of target release. Quantitative analysis of SMD-based target release kinetics in comparison to control cases utilizing (i) non-complementary CP-DP sequence and (ii) no DP. Average of 3 separate experiments is shown. Error bars indicate one standard deviation.

FIG. 4: Comparison between SMD-based and conventional multi-target sorting. (a) Rapid multi-target SMD-based sorting. Immunomagnetic isolation of 3 targets (red, green, and blue beads) retains targets within MB-bound fraction, leaving “impurity” (yellow beads) in supernatant. Sequential introduction of target-specific DPs leads to selective release of a target into supernatant, yielding pure unbound target fraction and leaving non-displaced targets in MB-bound fraction. (b) Multi-target sorting using sequential streptavidin-biotin bond-mediated target capture. 4-color bead sorting was done using antigen recognition by biotinylated antibodies followed by magnetic isolation of individual targets via streptavidin-biotin bond formation with streptavidin-coated MBs. Average value and standard deviation of 3 independent experiments is reported.

FIG. 5: SMD-based sorting selectivity at varying target abundance. Samples were prepared with test-to-reference bead ratio of 1:1, 1:5, 1:20, and 1:100 (holding concentration of reference and MBs constant). (a) Purity of target (blue beads) captured from a mix of Blue/Red beads in solution. (b) Purity of target (blue beads) released from a MB-bound fraction containing Blue/Red beads at different ratios. Purity of isolated test target fraction was determined by flow cytometry, with an average value and standard deviation of 3 independent experiments reported.

### DETAILED DESCRIPTION OF THE INVENTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2^{nd} Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

As used herein, the singular forms “a”, “an,” and “the” include plural referents unless the context clearly dictates otherwise. “And” as used herein is interchangeably used with “or” unless expressly stated otherwise.

Other than in the operating examples, or where otherwise indicated, all numbers expressing quantities of ingredients or reaction conditions used herein should be understood as modified in all instances by the term “about.” The term “about” when used in connection with percentages may mean±5% of the value being referred to. For example, about 100 means from 95 to 105.

All embodiments of any aspect of the invention can be used in combination, unless the context clearly dictates otherwise.

In a first aspect, the present invention provides methods for target isolation, comprising
(a) generating a first complex in a sample, wherein the first complex comprises:
   (i) a first single stranded nucleic acid (NA1);
   (ii) a second single stranded nucleic acid (NA2) bound to a surface, wherein the NA2 is complementary to the NA1, wherein the NA2 is bound to the NA1 via base pairing to form an NA1-NA2 complex comprising a double stranded region of at least 7 base pairs;
   (iii) a first binding molecule (B1) bound to the NA1; and
   (iv) a first target (T1) from the sample bound to the binding molecule; wherein the first complex comprises a T1-B1-NA1-NA2 complex;
(b) contacting the T1-B1-NA1-NA2 complex with a third single stranded nucleic acid (NA3); wherein the NA3 is complementary to a contiguous portion of either the NA1 or NA2 present in the double stranded region, such that hybridization of the NA3 to either the NA1 or NA2 is thermodynamically more favorable than the NA1-NA2 double stranded region; wherein the contacting occurs for a time and under conditions suitable to promote base pairing between the NA3 and either the NA1 and the NA2 to form a double stranded NA1-NA3 or NA2-NA3 region, whereby the NA1-NA2 double stranded region is disrupted; and
(c) isolating the T1 from the sample.

As disclosed in the examples that follow, the inventors have surprisingly discovered that the methods of the invention serve to quickly sort one or multiple targets in high yield and purity using selectively displaceable nucleic acid linkers. The methods can be combined with any suitable technique for nucleic acid separation to serve as a high throughput platform for sorting a wide variety of targets from a sample of interest.

NA1 (also referred to herein as an “encoding probe” or “EP”) and NA2 (also referred to herein as a “capture probe” or “CP”) share a region of complementarity that permits base pairing, resulting in a double stranded region that is at least 7 base pairs. NA3 (also referred to herein as a “displacement probe” or “DP”) is complementary to a contiguous portion of either the NA1 or NA2 present in the double stranded region after formation of the T1-B1-NA1-NA2 complex. NA3 is complementary to a contiguous portion of either NA1 or NA2 present in the double stranded region such that hybridization of the NA3 to either the NA1 or NA2 is thermodynamically more favorable than the NA1-NA2 double stranded region. Since displacement of the NA1-NA2 complex only happens when NA3 binding is thermodynamically favored. The binding of NA1-NA2 provides a means to rapidly and specifically form a complex (the T1-B1-NA1-NA2 complex) that contains a binding molecule bound to a target from a sample of interest, which is tethered to a surface via the binding of NA2 to the solid surface, thus permitting enrichment of the complex via any suitable means for capture of the surface. Use of the NA3 displacement probe permits rapid and specific disruption of the T1-B1-NA1-NA2 complex, such that the target can be rapidly isolated.

Each of NA1, NA2, and NA3 can be of any suitable length and any suitable nucleotide composition for a given purpose. Those of skill in the art will be able to design appropriate lengths and nucleotide composition of NA1, NA2, and NA3 based on the teachings herein. Generally, the higher the temperature and/or lower the salt concentration used, the longer the nucleic acids will need to be to provide specificity and stability sufficient for a given assay.

In one embodiment, NA1, NA2, and NA3 can be independently from about 7 to about 50 nucleotides in length or more. For example, each of NA1, NA2, and NA3 can be independently about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length. As will be understood by those of skill in the art, each of NA1, NA2, and NA3 may have additional nucleotides not involved in any base pairing interactions with each other. For example, one or more of NA1, NA2, or NA3 may include additional spacer nucleotides for some uses. In some embodiments, at least NA3 and one of NA1 and NA2 is about 16 nucleotides in overlap length. In some embodiments, at least NA3 and one of NA1 and NA2 is about 22 nucleotides in overlap length.

NA1 and NA2 hybridize over at least part of their length to form a double-stranded region of at least 7 base pairs. The length of the double stranded region can be any suitable length for a given assay, including but not limited to 7, 8, 9, 10, 11, 12, 13, 14, 15, 16, 17, 18, 19, 20, 21, 22, 23, 24, 25, 26, 27, 28, 29, or 30 base pairs. Those of skill in the art will understand, based on the teachings herein, the length of the double stranded region most appropriate for a given assay, in light of desired conditions including but not limited to temperature and salt concentration. For example, NA1 and NA2 can hybridize to form a double-stranded region that is stable at low temperatures but is unstable at high temperatures, i.e., the double-stranded region has a high melting temperature (Tm). For example, NA1 and NA2 can hybridize to form a double-stranded region having a Tm of 10° C. or higher (e.g., 10° C., 11° C. 12° C. 13° C., 14° C. 15° C., 16° C., 17° C., 18° C., 19° C. 20° C., 25° C. 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C. or higher). One of skill in the art is well aware of computational and experimental methods for determining the melting temperature of double stranded nucleic acids.

NA1 and NA2 can hybridize to form a double-stranded region of about 7 to about 30 consecutive base-pairs. However, the double-stranded region can be interrupted by one or more (e.g., one, two, three or more) single-stranded nucleotides in one or both of the strands. When both strands comprise single-stranded nucleotide(s) in the double-stranded region, they can be opposite to each other (i.e., forms a mismatch) or not next to each other (i.e., forms a bulge, loop, or hairpin). In one embodiment, the NA1-NA2 double strand region is not interrupted by any single-stranded nucleotides.

In various embodiments, NA1-NA2 can hybridize to form a double-stranded region of about 8-30, 8-29, 8-28, 8-27, 8-26, 8-25, 8-24, 8-23, 8-22, 8-21, 8-20, 8-19, 8-18, 8-17, 8-16, 8-15, 8-14, 8-13, 8-12, 8-11, 8-10, 8-9, 10-30, 10-29, 10-28, 10-27, 10-26, 10-25, 10-24, 10-23, 10-22, 10-21, 10-20, 10-19, 10-18, 10-17, 10-16, 10-15, 10-14, 10-13, 10-12, 10-11, 12-30, 12-29, 12-28, 12-27, 12-26, 12-25, 12-24, 12-23, 12-22, 12-21, 12-20, 12-19, 12-18, 12-17, 12-16, 12-15, 12-14, 12-13, 14-30, 14-29, 14-28, 14-27, 14-26, 14-25, 14-24, 14-23, 14-22, 14-21, 14-20, 14-19, 14-18, 14-17, 14-16, 14-15, 16-30, 16-29, 16-28, 16-27, 16-26, 16-25, 16-24, 16-23, 16-22, 16-21, 16-20, 16-19, 16-18, 16-17, 18-30, 18-29, 18-28, 18-27, 18-26, 18-25, 18-24, 18-23, 18-22, 18-21, 18-20, 18-19, 20-30, 20-29, 20-28, 20-27, 20-26, 20-25, 20-24, 20-23, 20-22, 20-21, 22-30, 22-29, 22-28, 22-27, 22-26, 22-25, 22-24, 22-23, 24-30, 24-29, 24-28, 24-27, 24-26, 24-25, 26-30, 26-29, 26-28, 26-27, 28-30, or 28-29 base pairs

Upon hybridization of NA1 and NA2, there can be a number of nucleotides of NA1 and/or NA2 not involved in the base pairing, which thus remain single stranded. In one embodiment, there is an “unbound region” of at least 1 nucleotide adjacent to (e.g.: directly next to) the double stranded region on either the NA1 or the NA2; this “unbound region” and contiguous nucleotides of NA1 or NA2 involved in the double stranded region are complementary to the NA3, thus permitting NA3 to disrupt the NA1-NA2 hybridization complex. As will be understood by those of skill in the art, the “unbound region” that is ultimately involved in base pairing with NA3 need not be at the 5′ or 3′ end of NA1 or NA2; it can be internal to the nucleic acid, or may be at the terminus, as deemed appropriate for a given assay. NA1 and NA2 can have any number of nucleotides not involved in NA1-NA2 base pairing, dictated by the length of NA1 and NA2 and the length of the double stranded region as deemed appropriate for a given assay. In various embodiments, the “unbound region” on either NA1 or NA2 that is involved in base pairing with the NA3 is between 1-25 nucleotides (e.g., 1, 2, 3, 4, 5, 6, 7, 8, 9, 10, 11, 12, 13, 14, 15, 16, 17, 18, 19, 20, 21, 22, 23, 24 or 25). In various embodiments, the “unbound region” on either NA1 or NA2 that is involved in base pairing with the NA3 is between 1-25, 2-25, 3-25, 4-25, 5-25, 6-25, 7-25, 8-25, 9-25, 10-25, 11-25, 12-25, 13-25, 14-25, 15-25, 16-25, 17-25, 18-25, 19-25, 20-25, 21-25, 22-25, 23-25, 24-25, 1-24, 2-24, 3-24, 4-24, 5-24, 6-24, 7-24, 8-24, 9-24, 10-24, 11-24, 12-24, 13-24, 14-24, 15-24, 16-24, 17-24, 18-24, 19-24, 20-24, 21-24, 22-24, 23-24, 1-23, 2-23, 3-23, 4-23, 5-23, 6-23, 7-23, 8-23, 9-23, 10-23, 11-23, 12-23, 13-23, 14-23, 15-23, 16-23, 17-23, 18-23, 19-23, 20-23, 21-23, 22-23, 1-22, 2-22, 3-22, 4-22, 5-22, 6-22, 7-22, 8-22, 9-22, 10-22, 11-22, 12-22, 13-22, 14-22, 15-22, 16-22, 17-22, 18-22, 19-22, 20-22, 21-22, 1-21, 2-21, 3-21, 4-21, 5-21, 6-21, 7-21, 8-21, 9-21, 10-21, 11-21, 12-21, 13-21, 14-21, 15-21, 16-21, 17-21, 18-21, 19-21, 20-21, 1-20, 2-20, 3-20, 4-20, 5-20, 6-20, 7-20, 8-20, 9-20, 10-20, 11-20, 12-20, 13-20, 14-20, 15-20, 16-20, 17-20, 18-20, 19-20, 1-19, 2-19, 3-19, 4-19, 5-19, 6-19, 7-19, 8-19, 9-19, 10-19, 11-19, 12-19, 13-19, 14-19, 15-19, 16-19, 17-19, 18-19, 1-18, 2-18, 3-18, 4-18, 5-18, 6-18, 7-18, 8-18, 9-18, 10-18, 11-18, 12-18, 13-18, 14-18, 15-18, 16-18, 17-18, 1-17, 2-17, 3-17, 4-17, 5-17, 6-17, 7-17, 8-17, 9-17, 10-17, 11-17, 12-17, 13-17, 14-17, 15-17, 16-17, 1-16, 2-16, 3-16, 4-16, 5-16, 6-16, 7-16, 8-16, 9-16, 10-16, 11-16, 12-16, 13-16, 14-16, 15-16, 1-15, 2-15, 3-15, 4-15, 5-15, 6-15, 7-15, 8-15, 9-15, 10-15, 11-15, 12-15, 13-15, 14-15, 1-14, 2-14, 3-14, 4-14, 5-14, 6-14, 7-14, 8-14, 9-14, 10-14, 11-14, 12-14, 13-14, 1-13, 2-13, 3-13, 4-13, 5-13, 6-13, 7-13, 8-13, 9-13, 10-13, 11-13, 12-13, 1-12, 2-12, 3-12, 4-12, 5-12, 6-12, 7-12, 8-12, 9-12, 10-12, 11-12, 1-10, 2-10, 3-10, 4-10, 5-10, 6-10, 7-10, 8-10, 9-10, 1-9, 2-9, 3-9, 4-9, 5-9, 6-9, 7-9, 8-9, 1-8, 2-8, 3-8, 4-8, 5-8, 6-8, 7-8, 1-7, 2-7, 3-7, 4-7, 5-7, 6-7, 1-6, 2-6, 3-6, 4-6, 5-6, 1-5, 2-5, 3-5, 4-5, 1-4, 2-4, 3-4, 1-3, 2-3, or 1-2 nucleotides.

The amount of NA1:NA2 hybridization complexes formed can be less than amount of surface or binding molecules, but should preferably be at least equal to amount of target. The amount of NA1 is linked to the amount of binding molecule (described herein), and the amount of NA2 is linked to the amount and type of surface (described herein). The amount of binding molecule in the complex depends on its affinity to target and can be in great excess to target (described herein).

The ratio of NA1:NA2 combined for hybridization purposes can be 1:1. In one embodiment, NA2 is used in large excess for hybridization (for example, at least 100×, 1000×, 5000×, etc.), to help drive the reaction. In another embodiment, NA1 is used in large excess for hybridization (for example, at least 100×, 1000×, 5000×, etc.), to help drive the reaction. Preferred ratios of NA1 to NA2 depend on target size, concentration, geometry of capture surface, and the sequence of complex formation. In one embodiment, NA1 and NA2 are added to the sample at nanomolar concentrations or less.

The NA3 is complementary to a contiguous portion of either the NA1 or NA2 present in the double stranded region such that hybridization of the NA3 to either the NA1 or NA2 is thermodynamically more favorable than the NA1-NA2 double stranded region. Thus, hybridization with NA3 to either of NA1 or NA3 disrupts the NA1-NA2 double stranded region by strand displacement. As used herein, the term “strand displacement” refers to replacing one of the nucleic acid strands in a double-stranded nucleic acid by third strand. Generally, strand displacement works best when the displacing strand is complementary to a longer stretch of NA1 or NA2. This can be accomplished by having either the NA1 or NA2 possess an unbound region (as described above) that is complementary to NA3 (in addition to the complementarity within the double stranded region. In addition, or alternatively, NA3 may comprise one or more modifications that promote duplex stability. Exemplary such modifications include, but are not limited to, 2-amino-A; 2-thio U; 5-Me-thio-U; G-clamp (an analog of C having 4 hydrogen bonds); pseudo-uridine; 2′ modifications, e.g., 2′F; “locked” nucleic acids (LNA) in which the oxygen at the 2′ position is connected by (CH₂)ₙ, wherein n=1-4, to the 4′ carbon of the same ribose sugar, preferably n is 1 (LNA) or 2 (ENA); inter-sugar modifications, such as phosphorothioates. In another embodiment, wherein the NA-NA2 double stranded region contains one or more interruptions by single-stranded nucleotides, the NA3 may simply be complementary with NA1 or NA2 over the entire length of NA1 or NA2 present in the double stranded region.

NA3 should be at least as abundant as the probe it binds to (either NA1 or NA2), but preferably in the micromolar concentration range to drive fast diffusion and displacement kinetics. It will be understood by those of skill in the art that not all NA1 (on target) and not all NA2 (on surface) participate in NA1:NA2 complex formation. In one embodiment, NA3 is added in large excess to NA1/NA2 to help drive the reaction. For example, NA3 may be added to the sample at micromolar concentration, while NA1 and NA2 are present at nanomolar concentrations or less.

In one embodiment, the resulting NA1-NA3 or NA2-NA3 double stranded region is at least 7 base pairs in length. In various embodiments, the NA1-NA3 or NA2-NA3 double stranded region is 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 base pairs in length. In various further embodiments, the NA1-NA3 or NA2-NA3 double stranded region is between 8-50, 8-45, 8-40, 8-35, 8-30, 8-25, 8-20, 8-15, 8-14, 8-13, 8-12, 8-11, 8-10, 10-50, 10-45, 10-40, 10-35, 10-30, 10-25, 10-20, 10-15, 10-14, 10-13, 10-12, 10-11, 12-50, 12-45, 12-40, 12-35, 12-30, 12-25, 12-20, 12-15, 12-14, 12-13, 15-50, 15-45, 15-40, 15-35, 15-30, 15-25, 15-20, 15-19, 15-18, 15-17, 15-16, 20-50, 20-45, 20-40, 20-35, 20-30, 20-25, 20-24, 20-23, 20-22, 20-21, 25-50, 25-45, 25-40, 25-35, 25-30, 25-29, 25-28, 25-27, 25-26, 30-50, 30-45, 30-40, 30-35, 30-34, 30-33, 30-32, 30-31, 35-50, 35-45, 35-40, 35-39, 35-38, 35-37, 35-36, 40-50, 40-45, 40-44, 40-43, 40-42, 40-41, 45-50, 45-49, 45-48, 45-47, or 45-46 base pairs in length.

In another embodiment, the length of the NA1-NA3 or NA2-NA3 double stranded region is at least the length of the NA1-NA2 double stranded region. In various further embodiments, the length of the NA1-NA3 or NA2-NA3 double stranded region is at least the length of the NA1-NA2 double stranded region plus 1, 2, 3, 4, 5, 6, 7, 8, 9, 10, 11, 12, 13, 14, 15, 16, 17, 18, 19, 20, or more additional base pairs. A suitable length of increase in the length of the NA1-NA3 or NA2-NA3 double stranded region compared to the NA1-NA2 double stranded region for a given assay can be determined by those of skill in the art based on the teachings herein. In various further embodiments, the length of the NA1-NA3 or NA2-NA3 double stranded region is at least the length of the NA1-NA2 double stranded region plus between 1-25, 2-25, 3-25, 4-25, 5-25, 6-25, 7-25, 8-25, 9-25, 10-25, 11-25, 12-25, 13-25, 14-25, 15-25, 16-25, 17-25, 18-25, 19-25, 20-25, 21-25, 22-25, 23-25, 24-25, 1-24, 2-24, 3-24, 4-24, 5-24, 6-24, 7-24, 8-24, 9-24, 10-24, 11-24, 12-24, 13-24, 14-24, 15-24, 16-24, 17-24, 18-24, 19-24, 20-24, 21-24, 22-24, 23-24, 1-23, 2-23, 3-23, 4-23, 5-23, 6-23, 7-23, 8-23, 9-23, 10-23, 11-23, 12-23, 13-23, 14-23, 15-23, 16-23, 17-23, 18-23, 19-23, 20-23, 21-23, 22-23, 1-22, 2-22, 3-22, 4-22, 5-22, 6-22, 7-22, 8-22, 9-22, 10-22, 11-22, 12-22, 13-22, 14-22, 15-22, 16-22, 17-22, 18-22, 19-22, 20-22, 21-22, 1-21, 2-21, 3-21, 4-21, 5-21, 6-21, 7-21, 8-21, 9-21, 10-21, 11-21, 12-21, 13-21, 14-21, 15-21, 16-21, 17-21, 18-21, 19-21, 20-21, 1-20, 2-20, 3-20, 4-20, 5-20, 6-20, 7-20, 8-20, 9-20, 10-20, 11-20, 12-20, 13-20, 14-20, 15-20, 16-20, 17-20, 18-20, 19-20, 1-19, 2-19, 3-19, 4-19, 5-19, 6-19, 7-19, 8-19, 9-19, 10-19, 11-19, 12-19, 13-19, 14-19, 15-19, 16-19, 17-19, 18-19, 1-18, 2-18, 3-18, 4-18, 5-18, 6-18, 7-18, 8-18, 9-18, 10-18, 11-18, 12-18, 13-18, 14-18, 15-18, 16-18, 17-18, 1-17, 2-17, 3-17, 4-17, 5-17, 6-17, 7-17, 8-17, 9-17, 10-17, 11-17, 12-17, 13-17, 14-17, 15-17, 16-17, 1-16, 2-16, 3-16, 4-16, 5-16, 6-16, 7-16, 8-16, 9-16, 10-16, 11-16, 12-16, 13-16, 14-16, 15-16, 1-15, 2-15, 3-15, 4-15, 5-15, 6-15, 7-15, 8-15, 9-15, 10-15, 11-15, 12-15, 13-15, 14-15, 1-14, 2-14, 3-14, 4-14, 5-14, 6-14, 7-14, 8-14, 9-14, 10-14, 11-14, 12-14, 13-14, 1-13, 2-13, 3-13, 4-13, 5-13, 6-13, 7-13, 8-13, 9-13, 10-13, 11-13, 12-13, 1-12, 2-12, 3-12, 4-12, 5-12, 6-12, 7-12, 8-12, 9-12, 10-12, 11-12, 1-10, 2-10, 3-10, 4-10, 5-10, 6-10, 7-10, 8-10, 9-10, 1-9, 2-9, 3-9, 4-9, 5-9, 6-9, 7-9, 8-9, 1-8, 2-8, 3-8, 4-8, 5-8, 6-8, 7-8, 1-7, 2-7, 3-7, 4-7, 5-7, 6-7, 1-6, 2-6, 3-6, 4-6, 5-6, 1-5, 2-5, 3-5, 4-5, 1-4, 2-4, 3-4, 1-3, 2-3, or 1-2 additional base pairs.

The double stranded NA1-NA3 or NA2-NA3 region can be interrupted by one or more (e.g., one, two, three or more) single-stranded nucleotides in one or both of the strands. When both strands comprise single-stranded nucleotide(s) in the double-stranded region, they can be opposite to each other (i.e., forms a mismatch) or not next to each other (i.e., forms a bulge, loop, or hairpin). In one embodiment, the double stranded NA1-NA3 or NA2-NA3 region is not interrupted by any single-stranded nucleotides.

As used herein, the term “nucleic acid” refers to a polymer or oligomer of nucleotide or nucleoside monomers consisting of naturally occurring bases, sugars and inter-sugar linkages. The term “nucleic acid” also includes polymers or oligomers comprising non-naturally occurring monomers, or portions thereof, which function similarly. At a minimum, a nucleic acid useful in the methods and kits described herein for coupling a binding molecule and surface is capable of sequence specific hydrogen-bonded hybridization between complementary nucleic acid strands. A nucleic acid can be DNA, RNA or chimeric, i.e., comprising both deoxy- and ribo-nucleotides. In some embodiments, the nucleic acids may comprise at least one modification. Typical nucleic acid modifications can include one or more of: (i) alteration, e.g., replacement, of one or both of the non-linking phosphate oxygens and/or of one or more of the linking phosphate oxygens in the phosphodiester intersugar linkage; (ii) alteration, e.g., replacement, of a constituent of the sugar, e.g., at the 2′ position of the sugar; (iii) wholesale replacement of the phosphate moiety with “dephospho” linkers; (iv) modification or replacement of a naturally occurring base with a non-natural base; (v) replacement or modification of the ribose-phosphate backbone, e.g. peptide nucleic acid (PNA); (vi) modification of the 3′ end or 5′ end of the nucleic acid, e.g., removal, modification or replacement of a terminal phosphate group or conjugation of a moiety, e.g., conjugation of a ligand, to either the 3′ or 5′ end of nucleic acid; and (vii) modification of the sugar, e.g., six membered rings. In some embodiments, the nucleic acid is not modified relative to naturally-occurring nucleic acid molecules. The terms replacement, modification, alteration, and the like, as used in this context, do not imply any process limitation, e.g., modification does not mean that one must start with a reference or naturally occurring nucleic acid and modify it to produce a modified nucleic acid but rather “modified” simply indicates a difference from a naturally occurring molecule. In some embodiments, the modification is selected from the group consisting of nucleobase modifications, sugar modification, inter-sugar (or inter-nucleoside) linkage modifications, backbone modifications (or sugar-phosphodiester replacement), and any combinations thereof. Exemplary sugar modifications at the sugar moiety include but are not limited to, modifying the 2′ position of the sugar, such as 2′-O-Me (2′-O-methyl), 2′-O-MOE (2′-O-methoxyethyl), 2′-F, 2′-O-[2-(methylamino)-2-oxoethyl] (2′-O-NMA), 2′-S-methyl, 2′-O—CH₂-(4′-C) (LNA), 2′-O—CH₂CH₂-(4 ‘-C) (ENA), 2’-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), 2′-NH₂, and 2-SH; arabinose sugar; threose sugar; and acyclic sugar (e.g., glycol nucleic acids). Exemplary inter-sugar linkage and backbone modifications include, but are not limited to, replacing one or both of the non-bridging phosphate oxygen atoms in the intersugar linkage can be replaced by the following: S, Se, BR₃ (R is hydrogen, alkyl, aryl), C (i.e. an alkyl group, an aryl group, etc. . . . ), H, NR₂; replacing one or both of bridging oxygen, (i.e. oxygen that links the phosphate to the nucleoside), with nitrogen (bridged phosphoroamidates), sulfur (bridged phosphorothioates) or carbon (bridged methylenephosphonates); replacing the phosphate group with amides (for example amide-3 (3′—CH₂—C(═O)—N(H)-5′) and amide-4 (3′—CH₂—N(H)—C(═O)-5)), hydroxylamino, siloxane (dialkylsiloxxane), carboxamide, carbonate, carboxymethyl, carbamate, carboxylate ester, thioether, ethylene oxide linker, sulfide, sulfonate, sulfonamide, sulfonate ester, thioformacetal (3′-S—CH₂—O-5′), formacetal (3 ‘-O—CH₂—O-5’), oxime, methyleneimino, methykenecarbonylamino, methylenemethylimino (MMI, 3′—CH₂—N(CH₃)—O-5′), methylenehydrazo, methylenedimethylhydrazo, methyleneoxymethylimino, ethers (C3′-O—C5′), thioethers (C3′-S—C5′), thioacetamido (C3′-N(H)—C(═O)—CH₂—S—C5′), C3′-O—P(O)—O—SS—C5′, C3′—CH₂—NH—NH—C5′, 3′-NHP(O)(OCH₃)—O-5′ and 3′-NHP(O)(OCH₃)—O-5′; and replacing the phosphate linker and sugar by nuclease resistant nucleoside or nucleotide surrogates, such as morpholino, cyclobutyl, pyrrolidine, peptide nucleic acid (PNA), aminoethylglycyl PNA (aegPNA), and backnone-extended pyrrolidine PNA (bepPNA) nucleoside surrogates. While one or both strands of the nucleic acid linker can comprise ribonucleotides, in some embodiments it is preferred that a strand is not comprised of all ribonucleotides. For example, a strand can comprise one, two, three, four, five, six, seven, eight, nine, ten, or more ribonucleotides as long as there is at least one (e.g., one, two, three, four, five, six, seven, eight, nine, ten, or more) 2′-deoxynucleotides in the strand. If more than one ribonucleotide is present, they can be present consecutively, i.e., next to each other, or non-consecutively.

The binding molecule may be bound to the NA1 at any position on NA1. For example, the binding molecule can be bound to the 3′ or 5′ end of the NA1, or to any internal base or bases of NA1. Similarly, the surface can be bound at any position in the NA2. For example, the surface can be bound to the 3′ or 5′ end of the NA2 or to any internal base or bases of NA2. Generally, the binding molecule and the surface are bound to positions on NA1 and NA2, respectively, such that hybridizing NA1 and NA2 does not interfere with functioning of the binding molecule or the surface. Accordingly, in some embodiments, the binding molecule is bound to the 3′ terminus of NA1 and the surface is bound to the 3′ terminus of NA2. In other embodiments, the binding molecule is bound to the 5′ terminus of NA1 and the surface is bound to the 5′ terminus of NA2. One or both strands of the double-stranded region can comprise a modification. When both strands comprise a modification, such a modification can be the same or different.

The binding molecule or the surface can be linked to the respective NA1 and NA2 covalently or non-covalently. Accordingly, in some embodiments, the binding molecule and NA1 and/or the surface and the NA2 are covalently linked together using a non-nucleic acid linker. For example, the binding molecule and the NA1 can be covalently linked together via a linker selected from the group consisting of a bond, succinimidyl-4-(N-maleimidomethyl)cyclohexane-1-carboxylate (SMCC) linker, sulfo-SMCC linker, succinimidyl-6-hydrazino-nicotinamide (S-HyNic) linker, N-succinimidyl-4-formylbenzamide (S-4FB) linker, bis-aryl hydrazone bond (from S-HyNic/S-4FB reaction), zero-length peptide bond (between —COOH and —NH2 directly on affinity molecule and nucleic acid), two peptide bonds on a spacer (from cross-linking of two —NH2 groups), triazole bond (from “click” reaction), a phosphodiester linkage, a phosphothioate linkage, and any combination thereof.

Alternatively, the binding molecule and the NA1 and/or the surface and the NA2 can be non-covalently linked together via an adaptor molecule, in which the adaptor molecule binds non-covalently with the binding molecule and NA1 is conjugated (covalently or non-covalently) with the adaptor molecule, or the adaptor molecule binds non-covalently with the surface and NA2 is conjugated (covalently or non-covalently) with the adaptor molecule. For example, the NA1 and/or NA2 can be covalently linked to the adaptor molecule by a linker selected from the group consisting of a bond, succinimidyl-4-(N-maleimidomethyl)cyclohexane-1-carboxylate (SMCC) linker, sulfo-SMCC linker, succinimidyl-6-hydrazino-nicotinamide (S-HyNic) linker, N-succinimidyl-4-formylbenzamide (S-4FB) linker, bis-aryl hydrazone bond (from S-HyNic/S-4FB reaction), zero-length peptide bond (between —COOH and —NH2 directly on affinity molecule and nucleic acid), two peptide bonds on a spacer (from cross-linking of two —NH2 groups), triazole bond (from “click” reaction), a phosphodiester linkage, a phsophothioate linkage, and any combination thereof.

Methods of linking a nucleic acid covalently or non-covalently with another molecule are well known in the art and available to an ordinarily skilled artisan. For example, DNA conjugated antibodies prepared with a variety of covalent and non-covalent procedures have been described for bio-analytical methods (Bailey, R. C., et al., DNA-encoded antibody libraries: a unified platform for multiplexed cell sorting and detection of genes and proteins. Journal of the American Chemical Society, 2007. 129(7): p. 1959-67 and Lind, K. and M. Kubista, Development and evaluation of three real-time immuno-PCR assemblages for quantification of PSA. Journal of Immunological Methods, 2005. 304(1-2): p. 107-16). In a non-limiting example, an amine-functionalized NA1 can be covalently linked to a binding molecule comprising a free sulfhydryl group. One such covalent conjugation approach involves reduction of IgG followed by maleimide mediated reaction between pre-activated NA1 and a sulfhydryl group in the Fc region of an antibody. One benefit of this approach is the controlled stoichiometry and structure of antibody-NA1 bioconjugate. As reaction is limited to 1-2 sites on the Fc region, the preparation of antibodies with intact Fab antigen recognition region and a single NA1 is readily achievable. This can be useful for minimizing potential off-target binding of antibody-NA1 bioconjugates.

Alternatively, amine cross-linking can be used for covalently linking NA1 to a binding molecule comprising an amine group. In some embodiments, the binding molecule and/or surface molecule can be non-covalently linked to NA1 and/or NA2, respectively, via an adaptor molecule to which the nucleic acid is linked, covalently or non-covalently. An adaptor molecule can be covalently linked to the nucleic acid using a linker. As used herein, the term “adaptor molecule” means any molecule that is capable of specifically binding with a binding molecule or surface. Generally, an appropriate adaptor molecule does not inhibit or reduce binding of the surface to NA2, or binding molecule to the target, i.e., binding of the binding molecule to its target is reduced very little, if at all, when said binding molecule is bound by an adaptor molecule. In one sense, an adaptor molecule can be a binding molecule as that term is used herein. Exemplary adaptor molecules include, but are not limited to protein A, protein G, antibody, portion of an antibody, antigen, receptor ligand, receptor, ligand binding fragment of a receptor, one member of a coupling pair, an aptamer, and the like. In some embodiments, when the binding molecule is an antibody or antibody derivative, the adaptor molecule is protein A, streptavidin, avidin, biotin, an antibody, or a portion of an antibody. In some embodiments, the adaptor molecule is not protein A, streptavidin, avidin, biotin, an antibody, or a portion of an antibody.

The target to be isolated can be any suitable target for which an appropriate binding molecule is available. In various non-limiting embodiments, the target may be one or more of cells of any kind (eukaryotic or prokaryotic), viruses, bacteria, proteins, polypeptides, nucleic acids, lipids, and carbohydrates, and other biological molecules or organisms.

The sample can be any sample from which a target is to be identified, including but not limited to genomic DNA, cell lysates, tissue homogenates, forensic samples, environmental samples, food samples, drug samples, a patient tissue sample (such as including but not limited to blood, serum, bone marrow, saliva, sputum, throat washings, tears, urine, semen, and vaginal secretions or surgical specimen such as biopsy or tumor, or tissue removed for cytological examination), research samples (including but not limited to cell extracts, tissue extracts, organ extracts, etc.), or any other sample of interest.

The binding molecule can be any suitable binding molecule for the target of interest that binds the target with adequate specificity to specifically bind the target in a complex mixture, and with sufficient avidity to maintain binding during the various method steps. As used herein, the term “specifically binding” and “specific binding” means that a binding molecule binds to the target with greater affinity than it binds to other molecules under the same conditions. Specific binding is generally indicated by a dissociation constant of 1 μM or lower, e.g., 500 nM or lower, 400 nM or lower, 300 nM or lower, 250 nM or lower, 200 nM or lower, 150 nM or lower, 100 nM or lower, 50 nM or lower, 40 nM or lower, 30 nM or lower, 20 nM or lower, 10 nM or lower, or 1 nM or lower.

Generally, the nature of interaction or binding between the binding molecule and the target molecule is non-covalent, such as one or more of hydrogen bonding, Van der Waals forces, electrostatic forces, hydrophobic forces, and the like. However, interaction or binding can also be covalent. The binding molecule may bind the target directly or indirectly (as part of a binding pair, which together bind the target). In various non-limiting embodiments, the binding molecule may comprise a antibody or aptamer (such as an antibody or aptamer against a molecular target, or an antibody or aptamer directed against a surface molecule on a cell or virus)

The binding molecule can be a naturally-occurring, recombinant or synthetic molecule. However, the binding molecule need not comprise an entire naturally occurring molecule but can consist of only a portion, fragment or subunit of a naturally or non-naturally occurring molecule. Exemplary binding molecules include, but are not limited to, ligand receptors, ligands for a receptor, one member of a coupling pair, nucleic acids (e.g., aptamers), peptides, proteins, peptidomimetics, antibodies, a portion of an antibody, antibody-like molecules, antigens, and the like. In some embodiments, the binding molecule is an antibody or a portion thereof. In some embodiments, the binding molecule is an antigen binding fragment of an antibody. As used herein, the term “antibody” or “antibodies” refers to an intact immunoglobulin or to a monoclonal or polyclonal antigen-binding portion with the Fc (crystallizable fragment) region or FcRn binding fragment of the Fc region. The term “antibodies” also includes “antibody-like molecules”, such as portions of the antibodies, e.g., antigen-binding portions. Antigen-binding portions can be produced by recombinant DNA techniques or by enzymatic or chemical cleavage of intact antibodies. “Antigen-binding portions” include, inter alia, Fab, Fab′, F(ab′)2, Fv, dAb, and complementarity determining region (CDR) fragments, single-chain antibodies (scFv), single domain antibodies, chimeric antibodies, diabodies, and polypeptides that contain at least a portion of an immunoglobulin that is sufficient to confer specific antigen binding to the polypeptide. Linear antibodies are also included for the purposes described herein. The terms Fab, Fc, pFc′, F(ab′) 2 and Fv are employed with standard immunological meanings (Klein, Immunology (John Wiley, New York, N.Y., 1982); Clark, W. R. (1986) The Experimental Foundations of Modern Immunology (Wiley & Sons, Inc., New York); and Roitt, I. (1991) Essential Immunology, 7th Ed., (Blackwell Scientific Publications, Oxford)). Antibodies or antigen-binding portions specific for various antigens are available commercially from vendors such as R&D Systems, BD Biosciences, e-Biosciences and Miltenyi, or can be raised against these cell-surface markers by methods known to those skilled in the art. In some embodiments, the binding molecule is not protein A, streptavidin, avidin, or biotin.

A binding molecule can be generated by any method known in the art. For example, antibodies can be found in an antiserum, prepared from a hybridoma tissue culture supernatant or ascites fluid, or can be derived from a recombinant expression system, as is well known in the art. Fragments, portions or subunits of e.g., an antibody, receptor or other species, can be generated by chemical, enzymatic or other means, yielding for example, well-known (e.g., Fab, Fab′) or novel molecules. The present invention also contemplates that binding molecules can include recombinant, chimeric and hybrid molecules, such as humanized and primatized antibodies, and other non-naturally occurring antibody forms. Those skilled in the art will recognize that the non-limiting examples given above describing various forms of antibodies can also be extended to other binding molecules such that recombinant, chimeric, hybrid, truncated etc., forms of non-antibody molecules can be used in the methods and kits of the present invention.

The binding molecule can be bound to more than one NA1. For example, the binding molecule can be bound to 1, 2, 5, 10, 25, 50, 100, 500, 1000, or more NA1s. As will be understood by those of skill in the art, the number of NA1s bound to the binding molecule will depend in large part on the size and type of binding molecule used. It is well within the level of skill in the art to determine an appropriate amount of NA1 to be bound to a given binding molecule, for a given assay. When the binding molecule is bound with two or more NA1s, the NA1s can all be the same, all different, or some same and some different. For example, the binding molecule may be “encoded” with several unique NA1s where one is used for target capture and another for labeling with fluorophore.

The surface can also be bound to more than one NA2. For example, the surface can be conjugated with 1, 2, 5, 10, 25, 50, 100, 500, 1000, or more NA2s. As will be understood by those of skill in the art, the number of NA2s bound to the surface will depend in large part on the type of surface used. For example, one magnetic bead can be bound to thousands to millions of NA2s, whereas a single macroscopic porous column support can hold orders of magnitude more NA2. It is well within the level of skill in the art to determine an appropriate amount of NA2 to be bound to a given surface, for a given assay. When the surface is conjugated with two or more NA2s, the NA2s can all be the same, all different, or some same and some different, such as when the same surface is used for capture of multiple different targets (as described herein).

The methods of the invention are well suited for isolation of multiple targets from a given sample. Thus, in one embodiment, the methods further comprise
(d) generating a second complex in the sample, wherein the second complex comprises:
   (i) a first single stranded nucleic acid (NA4);
   (ii) a second single stranded nucleic acid (NA5) bound to a surface, wherein the NA5 is complementary to the NA4, wherein the NA5 is bound to the NA4 via base pairing to form an NA4-NA5 complex comprising a double stranded region of at least 7 base pairs;
   (iii) a second binding molecule (B2) bound to the NA4; and
   (iv) a second target (T2) from a sample of interest bound to the binding molecule; wherein the complex comprises a T2-B2-NA4-NA5 complex;
(e) contacting the T2-B2-NA4-NA5 complex with a third single stranded nucleic acid (NA6); wherein the NA6 is complementary to a contiguous portion of either the NA4 or NA5 present in the double stranded region, such that hybridization of the NA6 to either the NA4 or NA5 is thermodynamically more favorable than the NA4-NA5 double stranded region; wherein the contacting occurs for a time and under conditions suitable to promote base pairing between the NA6 and either the NA4 or the NA5, whereby the NA4-NA5 double stranded region is disrupted; and
(f) isolating the T2 from the sample.

This embodiment permits simultaneous binding of target in the sample, and rapid/selective/specific isolation of multiple targets from the sample. All embodiments, combinations of embodiments, and definitions described above are applicable to the multiplex embodiments of the invention. In this embodiment, a second complex is generated, wherein NA1, NA2, and NA3 are replaced by NA4, NA5, and NA6, respectively. In this embodiment, any contiguous overlap between the NA4-NA5 double stranded region and the NA1-NA2 double stranded region must be short enough so that any cross-hybridization between the two complexes is disfavored. In one embodiment, any region of contiguous complementarity between the NA4-NA5 double stranded region and the NA1-NA2 double stranded region must be at least 1 nucleotide shorter than the length of either double stranded region. Similarly, any contiguous overlap between the NA4-NA6 or NA5-NA6 double stranded region and the NA1-NA3 or NA2-NA3 double stranded region must be short enough so that any cross-hybridization between the two complexes is disfavored. In one embodiment, any region of contiguous complementarity between the NA4-NA6 or NA5-NA6 double stranded region and the NA1-NA3 or NA2-NA3 double stranded region must be at least 1 nucleotide shorter than the length of either double stranded region. Those of skill in the art are well aware of oligonucleotide design programs to facilitate the production of large numbers of single stranded oligonucleotides with the requisite features required by the methods of this embodiment of the invention. In this embodiment, the binding molecule (B2), the target (T2), and the surface (S2) in the second complex may be the same or different as for the first complex. For example, the first and second target may be the same, while the first and second binding molecules may differ. This embodiment may be useful, for example, when targeting different epitopes on a given target. In another embodiment, the second surface may be the same as the first surface, to facilitate simultaneous enrichment of all complexes. In other embodiments, the second binding molecule and the second target are different than the first binding molecule and the first target.

In one embodiment, the first complex is contacted with NA3 to disrupt the first complex and the first target is isolated, followed by contacting of the sample with NA6 to disrupt the second complex and the second target is then isolated, in a sequential method. In another embodiment, the sample is contacted with NA3 and NA6 simultaneously, and the first and second target are then isolated. As will be understood by those of skill in the art, the methods can comprise generating third, fourth, fifth, sixth, seventh, or more complexes using the same methodology. Thus, in another embodiment, a method for multiplexed target isolation comprises
(a) generating a plurality of unique complexes in a sample, wherein each unique complex comprises:
   (i) a unique first single stranded nucleic acid (UNA1);
   (ii) a unique second single stranded nucleic acid (UNA2) bound to a surface, wherein the UNA2 is complementary to the UNA1, wherein the UNA2 is bound to the UNA1 via base pairing to form an UNA1-UNA2 complex comprising a double stranded region of at least 7 base pairs;
   (iii) a unique binding molecule (UB) bound to the UNA1; and
   (iv) a unique target (UT) from a sample of interest bound to the UB; wherein each unique complex comprises a UT-UB-UNA1-UNA2 complex;
(b) sequentially contacting each UT-UB-UNA1-UNA2 complex with a unique third single stranded nucleic acid (UNA3); wherein the UNA3 is complementary to a contiguous portion of either the UNA1 or UNA2 present in the double stranded region such that hybridization of the UNA3 to either the UNA1 or UNA2 is thermodynamically more favorable than the UNA1-UNA2 double stranded region; wherein the contacting occurs for a time and under conditions suitable to promote base pairing between the UNA3 and either the UNA1 or the UNA2, whereby the UNA1-UNA2 double stranded region is disrupted; and
(c) isolating each unique target from the sample.

In this embodiment, a series of unique complexes are generated, that differ from each other in at least the nucleic acids. As noted above, the unique binding molecule, the unique target, and the surface in each unique complex may be the same or different as for any one or more other different unique complexes. In one embodiment, the surface is the same in each unique complex, to facilitate simultaneous enrichment of all complexes. In other embodiments, the binding molecules and the targets are different in each unique complex. In various embodiments, the method comprises generating 2, 3, 4, 5, 6, 7, 8, 9, 10, 11, 12, 13, 14, 15, 16, 17, 18, 19, 20, or more unique complexes.

In one embodiment, a first unique UT-UB-UNA1-UNA2 complex is sequentially contacted with its UNA3 to disrupt one unique complex and isolating its target, followed by contacting of the sample the UNA3 of a second unique complex to disrupt the second unique complex and isolating its target, etc. In another embodiment, the sample is contacted with two or more of the UNA3s simultaneously, and the two or more unique targets are then isolated

The complexes (such as the T1-B1-NA1-NA2 complex) can be generated via any suitable means as deemed appropriate for a given assay. In one embodiment, generating the T1-B1-NA1-NA2 complex comprises:
(A) contacting the sample of interest with the NA1 bound to the B1; wherein the contacting occurs for a time and under conditions suitable for binding of the B1 to the T1 in the sample to form a T1-B1-NA1 complex;
(B) contacting the NA2 with the surface for a time and under conditions suitable to promote NA2 binding to the surface, thereby forming a surface-bound NA2;
(C) contacting the T1-B1-NA1 complex with the surface-bound NA2; wherein the contacting occurs for a time and under conditions suitable to promote formation of the NA1-NA2 complex, thereby forming the T1-B1-NA1-NA2 complex.

In another embodiment, generating the T1-B1-NA1-NA2 complex comprises:
(A) contacting the NA2 with the surface for a time and under conditions suitable to promote NA2 binding to the surface, thereby forming a surface-bound NA2;
(B) contacting the NA1 bound to the B1 with the surface-bound NA2; wherein the contacting occurs for a time and under conditions suitable to promote formation of the NA1-NA2 complex, thereby forming a B1-NA1-NA2 complex; and
(C) contacting the B1-NA1-NA2 complex with the sample of interest, wherein the contacting occurs for a time and under conditions suitable for binding of the B1 to the T1 in the sample to form the T1-B1-NA1-NA2 complex

In a further embodiment, generating the T1-B1-NA1-NA2 complex comprises:
(A) contacting the sample of interest with the NA1 bound to the B1; wherein the contacting occurs for a time and under conditions suitable for binding of the B1 to the T1 in the sample to form a T1-B1-NA1 complex;
(B) contacting the T1-B1-NA1 complex with the NA2; wherein the contacting occurs for a time and under conditions suitable to promote formation of the NA1-NA2 complex; and
(C) contacting the T1-B1-NA1-NA2 complex with the surface for a time and under conditions suitable to promote NA2 binding to the surface, thereby forming the complex.

In another embodiment, the methods can be carried out cyclically to enrich a target from the sample. This embodiment is particularly useful when the target is relatively rare in the sample. In this embodiment steps (a) (generating the T1-B1-NA1-NA2 complex) and (b) contacting the T1-B1-NA1-NA2 complex with NA3 to disrupt NA1-NA2 hybridization) are carried out two or more times. For example, if the first round of target isolation only yields a target purity of 50%, repeating the process by capturing and releasing the same target again could yield a target purity of 99%. In one embodiment, the displacement probe (NA3) binds the nucleic acid bound to the surface (NA2) and releases the T1-B1-NA1 complex, which would make the T1-B1-NA1 complex available for a second round of capture using same NA2; this process can be repeated as many times as appropriate for a given assay. Alternatively, the cycles could involve use of different binding molecules for a target, such as binding molecules directed to different surface epitopes of a single target.

Those of skill in the art will recognize that the same variations can be applied to generating any of the complexes disclosed herein.

Conditions and times suitable for hybridization between the different nucleic acids can routinely be determined by those of skill in the art, based on, for example, the nucleic acid length, specific nucleotide sequence used, concentration of the nucleic acids, temperature to be employed, salt conditions to be employed, etc. Similarly, suitable conditions for binding of the binding molecule to target present in a sample can routinely be determined by those of skill in the art, based on, for example, the volume of the sample, expected abundance of the target in the sample, the specific binding molecule and target and the nature of their interaction, etc. Exemplary conditions are provided in the examples that follow.

As used therein, the term “contacting” refers to any suitable means for delivering, or exposing, to a sample, the specified composition or molecule. In some embodiments, the term “contacting” refers to adding specific composition or molecules (e.g., suspended in a solution) directly to the sample. In some embodiments, the term “contacting” can further comprise mixing the sample with the specific composition or molecules by any means known in the art (e.g., vortexing, pipetting, and/or agitating). In some embodiments, the term “contacting” can further comprise incubating the sample together with the specific composition or molecules for a sufficient amount of time, e.g., to allow binding of the binding molecule to the target, or base pairing between the nucleic acids. The contact time can be of any suitable length, depending on the binding affinities and/or concentrations of the binding molecules or the targets, concentrations of the binding molecules, length, base content, and concentration of the nucleic acids, or incubation condition (e.g., temperature). In some embodiments, the contact time between the sample containing target and the binding molecule, and/or the nucleic acids can be at least about 30 seconds, at least about 1 minute, at least about 5 minutes, at least about 10 minutes, at least about 15 minutes, at least about 30 minutes, at least about 1 hour, at least about 2 hours, at least about 3 hours, at least about 4 hours, at least about 6 hours, at least about 8 hours, at least about 10 hours, at least about 12 hours, at least about 24 hours, at least about 48 hours or longer. One of skill in the art can adjust the contact time and conditions accordingly.

The binding of NA1-NA2 provides a means to rapidly and specifically form a complex (the T1-B1-NA1-NA2 complex) that contains a binding molecule bound to a target from a sample of interest, which is tethered to a surface via the binding of NA2 to the solid surface, thus permitting enrichment of the complex via any suitable means for capture of the surface. Use of the NA3 displacement probe permits rapid and specific disruption of the T1-B1-NA1-NA2 complex, such that the target can be rapidly isolated. Any suitable method for isolation can be employed; the specific isolation technique employed will depend on the surface employed, the target type (cells, vs. proteins, etc.) and concentration, etc. Such isolation techniques will be readily apparent to those of skill in the art based on the teachings herein For example, the methods can further comprise any suitable method of enrichment of the complex prior to isolation, such as concentration of the complex via selection of the surface. In one embodiment, the surface comprises a magnetic particle, and the method may comprise applying a magnetic field to the sample to attract the magnetic particles and thus to concentrate the complex prior to disruption by use of the NA3 and isolation of the target. In other embodiments, the surface comprises a particle that can be centrifuged under conditions that do not result in complex dissociation, thereby concentrating the complex prior to disruption by use of the NA3 and isolation of the target. It will be readily apparent to those of skill in the art based on the teachings herein how to modify the enrichment/isolation approaches based on the specific surface used and the identity of the complex components and specific conditions to be employed for a given assay.

As used herein, “isolating” means to separate the target from the sample and to thus concentrate it; it does not require that the target is isolated in pure form. In one embodiment the target is substantially purified from the sample; in another embodiment, the target is partially purified from the sample, such that it its concentration in the resulting “isolate” is at least 10×, 50×, 100×, or 200× greater than its concentration in the starting sample. In other embodiments, the isolate is at least 75% purified target; in further embodiments, at least 80%, 85%, 90%, 95%, 96%, 97%, 98%, 99%, or greater purified target.

The methods of the invention may comprise any further steps as suitable for a given assay. In some embodiments, the methods may comprise one or more washing or blocking steps. For example, a sample can be subjected to one or more blocking steps before contacting the sample with the binding molecule to reduce or inhibit non-specific binding of the binding molecule to the sample. One or more blocking steps can also be used before contacting the sample with the surface bound NA2 to reduce or inhibit non-specific binding of the surface to undesired binding molecule or the sample. One or more washing steps can be performed after a contacting step to wash away any leftover reagents from the contacting step. Such washing and blocking steps are well known in the art, and the specific conditions employed for a given blocking or washing step can readily be determined by one of skill in the art based on the teachings herein.

In a non-limiting embodiment, the methods involve incubation of the sample with a large excess of NA1-B (whether already in the complex with NA2 or NA2-S or not). The sample/target is incubated with a high ratio of NA1-B to drive the reaction, though the amount of NA1-B is generally not so great as to lead to excessive non-specific binding. Unbound NA1-B is then removed in one or more wash steps. If NA1-B was not already complexed with NA2, the T-B-NA1 complex is then incubated with an excess of NA2-S to drive the reaction; unbound NA2-S is then removed in one or more wash steps. The resulting T-B-NA1-NA2-S complex is then enriched by selection of the surface; for example, if the surface comprises a magnetic bead, a magnetic field is used to concentrate the T-B-NA1-NA2-S complex. The T-B-NA1-NA2-S complex is then incubated with an excess of NA3 to drive the strand displacement reaction to release the T-B-NA1 complex from the surface, thus isolating the target.

In another aspect, the invention provides kits comprising
(a) a first single stranded nucleic acid (NA1);
(b) a second single stranded nucleic acid (NA2) capable of binding to a surface, wherein the NA2 is complementary to the NA1, wherein the NA2 is capable of binding to the NA1 via base pairing to form an NA1-NA2 complex a double stranded region of at least 7 base pairs;
(c) a third single stranded nucleic acid (NA3); wherein the NA3 is complementary to a contiguous portion of either the NA1 or NA2 present in the double stranded region such that hybridization of the NA3 to either the NA1 or NA2 is thermodynamically more favorable than the NA1-NA2 double stranded region.

All embodiments and combinations of embodiments of the kit components disclosed above for the methods of the invention are equally applicable in the kits of the invention. In one embodiment, the NA1 is bound to a binding molecule, as described in the methods of the invention. NA2 is capable of binding to a surface via any suitable means. In one non-limiting embodiment, NA2 is modified with a linker for binding to the surface covalently or non-covalently. The surface may be any suitable surface, including but not limited to magnetic particles, microarrays, beads, columns, optical fibers, wipes, nitrocellulose, nylon, glass, quartz, diazotized membranes, silicones, polyformaldehyde, cellulose, cellulose acetate, paper, ceramics, metals, metalloids, semiconductive materials, coated beads, plastics, gel-forming materials, sol gels, porous polymer hydrogels, nanostructured surfaces, nanotubes, and nanoparticles.

The kit may comprise multiple unique NA1, NA2, and NA3s as described for the multiplex methods of the invention. The kit may further comprise wash and/or blocking reagents, and any other components disclosed herein that may be suitable for using the kits to carry out the methods of the invention.

To the extent not already indicated, it will be understood by those of ordinary skill in the art that any one of the various embodiments herein described and illustrated can be further modified to incorporate features shown in any of the other embodiments disclosed herein.

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### EXAMPLES

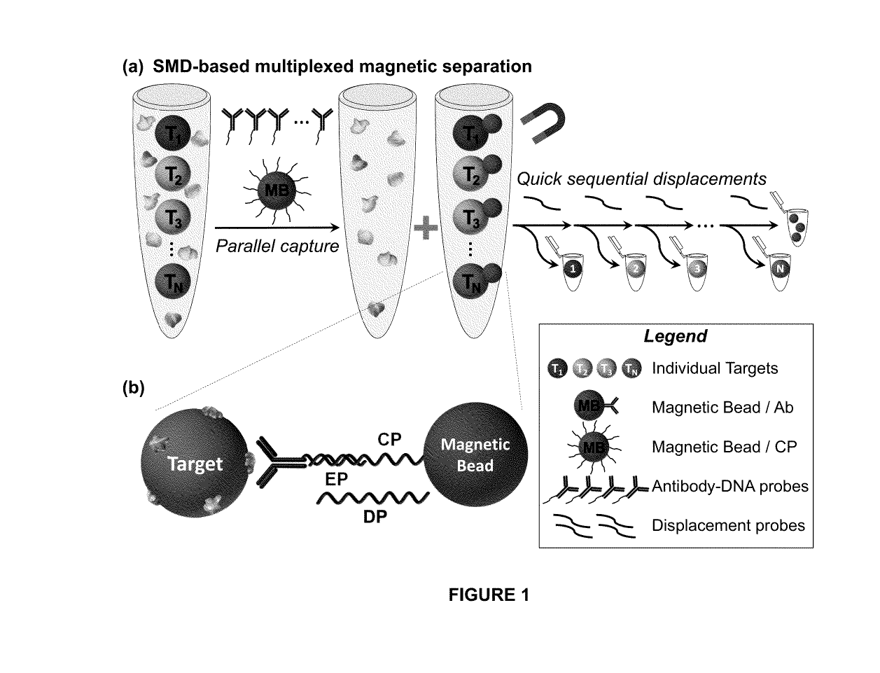

This report describes a rapid multi-target immunomagnetic sorting technology that combines extensive multiplexing capacity of DNA-antibody conjugates and high selectivity, throughput, and simplicity of magnetic isolation by employing a unique sorting approach through strand-mediated displacement (SMD) of DNA linkers. A key insight for this work was that multiplexing selection through specific DNA sequences could offer simultaneous selection of multiple target populations from a heterogeneous sample, followed by quick isolation of individual targets through SMD. The major steps of one embodiment of SMD for multi-target sorting are illustrated in FIG. 1a. In the first step, antibodies encoded with distinct DNA sequences (encoding probe, EP) bind their identifying antigen on target populations. Next, magnetic beads (MBs) coated with capture probes (CPs), which partially hybridize with their corresponding EPs, enable simultaneous magnetic selection of all targets of interest. In the second step, the magnetically enriched targets are released one population at a time through SMD upon addition of a displacement probe (DP). Probe assembly is schematically illustrated in FIG. 1b. The DP binds selectively to an exposed ‘toehold’ region on the CP, and then ‘unzips’ the EP from its original binding site due to the longer complementarity between CP and DP, thus breaking the MB-target link. In this way, multiple targets can be quickly isolated through a serial addition of DPs. In direct contrast to the conventional multi-cycle magnetic separation, SMD technology employs only a single round of slow immunorecognition, and the following SMDs are a remarkably rapid process (with a sub-second displacement half-time) [23]. Therefore, with sorting of N targets (N>1), the present methods offer significantly shortened assay time (N×Y hours vs. Y hours+rapid SMDs for assay time of Y hours), which is highly desired for preserving the native state and bio-functionality of isolated targets.

### Materials

Fluorescent beads (Bangs Laboratories) 5-6 μm in diameter doped with 4 different organic dyes (Glacial Blue 360/450 nm excitation/emission maxima, Dragon Green 480/520 nm, Suncoast Yellow 540/600 nm, and Flash Red 660/690 nm) were used as a model system for development and characterization of the SMD technology. Each bead features surface carboxylic acid functional groups (with parking area between 37 and 174 A²/surface group) suitable for covalent conjugation with biomolecules. Magnetic beads (Dynabeads MyOne Streptavidin C1, Invitrogen) are 1 μm in diameter and feature streptavidin coating for easy assembly with biotinylated DNA probes. Purified IgG from human, mouse, and rabbit serum as well as whole goat anti-human, anti-mouse, and anti-rabbit IgG were purchased from Sigma-Aldrich. All antibodies were obtained in 1×PBS without carrier proteins or stabilizing reagents. Biotinylated goat anti-human, anti-mouse, and anti-rabbit IgG were either purchased from Sigma-Aldrich or prepared in house using EZ-Link NHS-PEG4-Biotin (Thermo Scientific). DNA probes were purchased from Integrated DNA Technologies. Sequences were optimized to minimize secondary structures and homology with mismatched DNA sequences at room temperature. Encoding probes (EPs) were synthesized with primary amine functional group at the 5′ end for covalent conjugation with antibodies. Capture probes (CPs) were synthesized with a biotin tag at the 5′ end for assembly with streptavidin-coated MBs. Both CPs and EPs included a 5′ 10 A spacer to allow for flexibility at the bead interface. All DNA probes were purified with HPLC, reconstituted in DNAase-free water (Thermo Scientific) at 100 μM, and stored at −20° C. Sequences of DNA probes are summarized in Table 1.

TABLE 1
DNA sequences used for multi-target enrichment and isolation. To demonstrate
multi-target enrichment and isolation, 3 sets of DNA sequences were designed with 16 base-pair
overlap (shown in red) between CP and EP and a 6 base-pair toehold region, allowing for a total
of 22 base-pair DP binding. The toehold region is bolded. 10A spacer at the 5′ end of EP and CP
is in black.

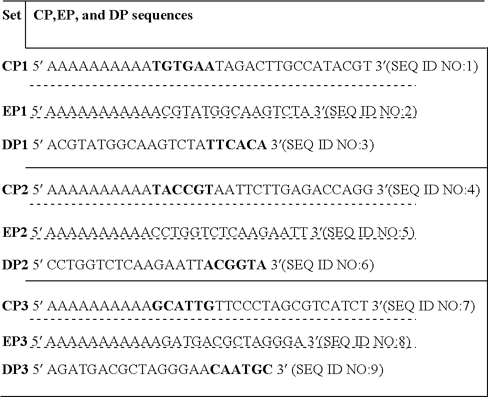

### Preparation of DNA-Antibody Conjugates

Functionalization of antibodies with encoding DNA sequences was achieved by covalent conjugation between primary amine groups present on antibody and the 5′-end primary amine group on DNA. First, IgG was activated with S-HyNic (succinimidyl-6-hydrazino-nicotinamide, Solulink) heterobifunctional cross-linker, which introduces aromatic hydrazine group: 100 μL 1 mg/mL IgG in 100 mM PBS was mixed with 2 μL 10 mM S-HyNic (in DMF) and incubated for 2 hours. At the same time, EP was activated with S-4FB (N-succinimidyl-4-formylbenzamide, Solulink) heterobifunctional cross-linker, which converts primary amine into aromatic aldehyde group: 100 μL 50 μM EP in 100 mM PBS was mixed with 2.5 μL 100 mM S-4FB (in DMF) and incubated for 2 hours. Excess cross-linkers was then removed by passing both activated IgG and EP through Zeba spin desalting columns (Thermo Scientific), and buffer was exchanged to 100 mM MES, pH5. Finally, activated IgG and EP were conjugated through formation of bis-arylhydrazone bond between aromatic hydrazine and aromatic aldehyde groups: IgG and EP were mixed together at ˜20× molar excess of EP, reacted for 4 hours, and buffer-exchanged into 10 mM PBS with Zeba spin desalting columns. All reactions were performed at room temperature. DNA-antibody conjugates were stored at 4° C. and used within 2 months after preparation.

### Preparation of Antigen-Coated Fluorescent Beads

IgGs purified from human, rabbit, and mouse serum were covalently linked to the surface of red, green, and blue fluorescent beads, respectively. Covalent conjugation was achieved via 2-step carbodiimide-mediated cross-linking between primary amines on IgG and carboxylic acid groups on bead surface. First, fluorescent beads were washed and suspended in MES buffer (pH 4.7) with 0.01% Tween-20 at 0.1 w/v % (˜10⁷ beads/mL) and activated for 15 minutes upon addition of 10 mg/mL 1-Ethyl-3-(3-dimethylaminopropyl)carbodiimide (EDC, Sigma-Aldrich) and 10 mg/mL N-hydroxysulfosuccinimide (sulfo-NHS, Thermo Scientific). Activated beads were washed by centrifugation (at 3000 g for 2 minutes) twice using 50 mM Borate buffer (pH 8.4) with 0.01% Tween-20 to remove excess reactants and then incubated with IgG at 2.5 mg/mL in Borate buffer with 0.01% Tween-20 for 4-8 hours for covalent cross-linking IgG-conjugated microspheres were washed 4 times to remove excess IgG, resuspended in 10 mM PBS with 0.5% Bovine Serum Albumin (BSA, Sigma-Aldrich), and stored at 4° C.

### Validation of Antigen Coating on the Surface of Fluorescent Beads

Presence of target-specific surface antigen (mouse, rabbit, or human IgG) on the surface of each fluorescent bead population was tested via labeling with biotinylated goat anti-mouse, anti-rabbit, or anti-human IgG followed by staining with quantum dot probes functionalized with streptavidin (Qdot 655 streptavidin conjugate, Invitrogen). PBS with 0.5% BSA was used as incubation and washing buffer throughout the experiment. All incubation steps were carried out at room temperature under gentle rotation. All washing steps were done through centrifugation at 3000 g for 2 minutes. Each bead type was resuspended in 4000 μL buffer at a final concentration of 1×10⁶ beads/mL and split into 4 test volumes 100 μL each. To 3 of the 4 samples, biotinylated IgGs were added at 0.2 μg/mL, incubated for 30 minutes, washed 3 times, and resuspended with 100 μL buffer. The forth sample (control) was incubated with only 0.5% BSA/PBS and washed in the same fashion. Then QDot655-streptavidin probes were added to each sample at 1 nM final concentration, incubated for 30 minutes, washed with buffer 4 times, and finally washed with water once. Pellets were resuspended in 10 μL water, spotted onto glass cover slips, allowed to dry, and imaged at high magnification. Wide UV filter cube was used for imaging of all fluorescent beads and quantum dots (as UV light provides sufficient excitation energy for all fluorophores). Hyper-spectral imaging and further image analysis with Nuance software enabled unmixing of fluorescence signal components and direct quantitative analysis of Qdot staining intensity on the surface of fluorescent beads. False-color composite images were obtained by merging individual channels.

### Study of Target Capture and Release Kinetics

Green fluorescent beads conjugated with rabbit IgG were used for the study of target capture and release kinetics. PBS with 0.5% BSA was used throughout all steps of this study. All incubation steps were performed under gentle rotation at room temperature unless noted otherwise. The conditions for capture experiments are outlined in Table 2. In step 1a, target beads were incubated with capture antibodies (with conjugated biotin or EP) for immunorecognition: 10⁶ fluorescent beads/mL were mixed with capture antibodies at final antibody concentration of 2.5 μg/mL in 100 μL buffer and incubated for 30 minutes. Meanwhile, in step 1b, biotinylated CPs were immobilized onto streptavidin-coated magnetic beads: 10⁷ MBs were mixed with CPs at final DNA concentration of 1 μM and incubated for 30 minutes. Fluorescent beads were washed through centrifugation (at 3000 g for 2 minutes) 4 times to remove excess antibody, while MBs were washed 4 times with a magnet to remove excess CPs. Fluorescent beads were then mixed with the magnetic fraction at a ratio of 50 MBs per fluorescent bead. For study of capture kinetics, the mixture was washed 3 times on the magnet following 5, 15, 30, and 60 minute incubation periods; supernatant fractions were pooled, and the MB-bound fraction was resuspended in equal volume. For study of target release kinetics, MB-bound fluorescent beads were prepared using the protocol described above (Condition 2 in Table 2). The conditions for SMD experiments are outlined in Table 3. Displacement probe (DP) was added to the mixture at 5 μM final concentration, and the solution was gently mixed for ˜30 seconds with pipette. MB-bound fraction was separated with a magnet after 1 minute and 60 minute incubation periods. For both capture and release studies, reproducibility was evaluated by conducting three independent trials. For fluorescence measurement samples were placed into a 96-well black flat-bottom plate (Corning). Fluorescence in MB-bound and supernatant fractions was measured with fluorescence plate reader (Infinite M200, Tecan) at 480/541 nm excitation/emission wavelengths and constant gain. Measurements were averaged over 4 quadrants of each well to correct for inhomogeneous sedimentation of fluorescent beads. Equal volume of PBS with 0.5% BSA was used as a baseline, which was subtracted from all fluorescence readings. Fraction captured was calculated as the baseline-corrected fluorescence in MB-bound fraction divided by the sum of the baseline-corrected fluorescence in MB-bound fraction and supernatant.

TABLE 2
Conditions for the study of target capture kinetics

| | Capture | |
|---|---|---|
| | antibody type | MB type |
| Condition 1: Biotin-streptavidin mediated | Goat anti- | Streptavidin |
| capture | Rabbit/Biotin | |
| Condition 2: Capture via complementary | Goat anti- | CP1 |
| DNA hybridization | Rabbit/EP1 | |
| Condition 3: Non-complementary DNA | Goat anti- | CP2 |
| hybridization | Rabbit/EP1 | |
| Condition 4: Mismatched antibody binding | Goat anti- | CP1 |
| | Mouse/EP1 | |
| Condition 5: Nonspecific binding | None | Streptavidin |

TABLE 3
Conditions for the study of target release kinetics

| | DP |
|---|---|
| Condition 1: Release through complementary DP | DP1 |
| Condition 2: Release through non-complementary DP | DP2 |
| Condition 3: Release with no SMD | None |

Study of Target Capture Specificity (from a 4-Bead Mix)

Three sets of “target” fluorescent beads were prepared: green beads were coated with rabbit IgG, blue beads with mouse IgG, and red beads with human IgG. Uncoated yellow beads were used as an “impurity” fraction. PBS with 0.5% BSA was used as incubation and washing buffer throughout this study. All incubation steps were performed under gentle rotation at room temperature. Purity of isolated fractions was quantitatively measured with flow cytometry (method described separately) and qualitatively evaluated with fluorescence microscopy (method described separately). Three independent trials for each experiment were conducted to evaluate reproducibility.

Part A (Specificity of Antibody-Antigen Recognition for Biotinylated IgGs):

Four bead populations were pooled in even proportions into a single centrifuge tube to a total final concentration of 4×10⁶ beads/mL and split into 4 test volumes 100 μL each. To 3 of the 4 samples, biotinylated Abs against rabbit, mouse, or human IgG were added at 0.2 μg/mL, incubated for 15 minutes, washed through centrifugation (at 3000 g for 2 minutes) 4 times to remove excess Abs, and resuspended with 100 μL buffer. The forth sample (control) was incubated with buffer only and washed in the same fashion. Next, fluorescent beads were mixed with streptavidin-coated MBs at a ratio of 50 MBs per target fluorescent bead and incubated for 30 minutes. Each sample was then separated on a magnet for enrichment and isolation of green, blue, or red beads from the initial 4-color mixture. The magnetic fraction was washed 3 times, the supernatant fractions were pooled together, volumes of magnetic and supernatant fractions were equalized, and the absolute number of fluorescent beads in each fraction was counted with flow cytometry. All conditions tested are summarized in Table 4.

TABLE 4
Conditions for the study of antibody-antigen binding specificity for
IgG-biotin

| | Capture | |
|---|---|---|
| | antibody type | MB type |
| Condition 1: Blue bead (Mouse IgG | Goat anti- | Streptavidin |
| coating) capture | Mouse/Biotin | |
| Condition 2: Green bead (Rabbit IgG | Goat anti- | Streptavidin |
| coating) capture | Rabbit/Biotin | |
| Condition 3: Red bead (Human IgG | Goat anti- | Streptavidin |
| coating) capture | Human/Biotin | |
| Condition 4: Control (non-specific MB | None | Streptavidin |
| binding) | | |

Part B (Specificity of Antibody-Antigen Recognition for DNA-Antibody Conjugates):

Four bead populations were pooled in even proportions into a single centrifuge tube to a total final concentration of 4×10⁶ beads/mL and split into 4 test volumes 100 μL each. To 3 of the 4 samples, DNA-antibody conjugates against rabbit, mouse, or human IgG were added at 2 μg/mL, incubated for 15 minutes, washed through centrifugation (at 3000 g for 2 minutes) 4 times, and resuspended with 100 μL buffer. The fourth sample (control) was incubated with buffer only and washed in the same fashion. Meanwhile, MBs with CPs (CP1, CP2, or CP3) were prepared by mixing 10⁷ MBs with CPs at final DNA concentration of 1 μM, incubating for 15 minutes, and washing 4 times with a magnet. The fluorescent beads were then mixed with CP-MBs complementary to the capture DNA-antibody conjugate at a ratio of 50 MBs per target fluorescent bead and incubated for 30 minutes. Each sample was then separated on a magnet for enrichment and isolation of green, blue, or red beads from the initial 4-color mixture. The magnetic fraction was washed 3 times, the supernatant fractions were pooled together, volumes of magnetic and supernatant fractions were equalized, and the absolute number of fluorescent beads in each fraction was counted with flow cytometry. All conditions tested are summarized in Table 5.

TABLE 5
Conditions for the study of antibody-antigen binding specificity for
IgG-DNA

| | Capture | |
|---|---|---|
| | antibody type | MB type |
| Condition 1: Blue bead (Mouse IgG | Goat anti-Mouse/ | CP2 |
| coating) capture | EP2 | |
| Condition 2: Green bead (Rabbit IgG | Goat anti-Rabbit/ | CP1 |
| coating) capture | EP1 | |
| Condition 3: Red bead (Human IgG | Goat anti- | CP3 |
| coating) capture | Human/EP3 | |
| Condition 4: Control (non-specific MB | None | Streptavidin |
| binding) | | |

Part C (Specificity of Complementary DNA Hybridization for EP-CP Oligonucleotide pairs):

Each “target” fluorescent bead type was separately labeled with its corresponding DNA-antibody conjugate. Fluorescent beads at a final concentration of 1×10⁶ beads/mL in a 100 μL test volume were mixed with 2 μg/mL IgG-DNA, incubated for 15 minutes, and washed through centrifugation (at 3000 g for 2 minutes) 4 times. Then the three “target” bead populations and “impurity” yellow beads were mixed together at even proportions to a total final concentration of 4×10⁶ beads/mL. This way, potential nonspecific antigen-antibody binding was circumvented, and specificity of oligonucleotide hybridization could be independently assessed. Meanwhile, MBs with CPs (CP1, CP2, or CP3) were prepared by mixing 10⁷ MBs with CPs at final DNA concentration of 1 μM and incubating for 15 minutes, and washing 4 times with a magnet. Magnetic beads with no CP were used as a control. The fluorescent beads were then mixed with CP-MBs corresponding to capture of a single target by DNA hybridization at a ratio of 50 MBs per target fluorescent bead and incubated for 30 minutes. Each sample was then separated on a magnet for enrichment and isolation of green, blue, or red beads from the initial 4-color mixture. The magnetic fraction was washed 3 times, the supernatant fractions were pooled together, volumes of magnetic and supernatant fractions were equalized, and the absolute number of fluorescent beads in each fraction was counted with flow cytometry. All conditions tested are summarized in Table 6.

TABLE 6
Conditions for the study of EP-CP hybridization specificity for IgG-DNA

| | Capture antibody type | MB type |
|---|---|---|
| Condition 1: Blue bead (Mouse | Goat anti-Rabbit/EP1 | CP2 |
| IgG coating) capture | Goat anti-Mouse/EP2 | |
| | Goat anti | |
| | Human/EP3 | |
| Condition 2: Green bead (Rabbit | Goat anti-Rabbit/EP1 | CP1 |
| IgG coating) capture | Goat anti-Mouse/EP2 | |
| | Goat anti- | |
| | Human/EP3 | |
| Condition 3: Red bead (Human | Goat anti-Rabbit/EP1 | CP3 |
| IgG coating) capture | Goat anti-Mouse/EP2 | |
| | Goat anti- | |
| | Human/EP3 | |
| Condition 4: Control (non-specific | Goat anti-Rabbit/EP1 | Streptavidin |
| MB binding) | Goat anti-Mouse/EP2 | |
| | Goat anti- | |
| | Human/EP3 | |

Study of Strand-Mediated Displacement Specificity for Target Release (from a 4-Bead Mix)

Three sets of “target” fluorescent beads were prepared for this experiment: green beads were coated with rabbit IgG, blue beads with mouse IgG, and red beads with human IgG. Uncoated yellow beads were used as an “impurity” fraction. PBS with 0.5% BSA was used as incubation and washing buffer throughout this study. All incubation steps were performed under gentle rotation at room temperature. Separately, each “target” bead type was labeled with its corresponding DNA-antibody conjugate. Fluorescent beads at a final concentration of 1×10⁶ beads/mL in a 100 μL test volume were incubated with 2 μg/mL DNA-antibody conjugate for 15 minutes, and washed through centrifugation (at 3000 g for 2 minutes) 4 times. Then the three “target” bead populations and “impurity” yellow beads were mixed together at even proportions to a total final concentration of 4×10⁶ beads/mL. This way, weak nonspecific antigen-antibody binding/unbinding was circumvented, and specificity of target release via SMD could be independently assessed. Meanwhile, MBs with CPs (CP1, CP2, and CP3) were prepared by mixing 3×10⁷ MBs with CPs at total final DNA concentration of 3 μM, incubating for 15 minutes, and washing on a magnet 4 times. For capture of all 3 targets, fluorescent beads were mixed with CP-MBs at a ratio of 50 MBs per target fluorescent bead and incubated for 30 minutes. Each sample was then separated on a magnet for enrichment and isolation of green, blue, or red beads from the initial 4-color mixture. The magnetic fraction was washed 3 times. Following magnetic capture, displacement probe corresponding to release of a single target (DP1: green, DP2: blue, DP3: red) was added to the sample at 5 μM final concentration, mixed gently with pipette for 1 minute, and then placed immediately on the magnet for collection of released beads in the supernatant. The sample was washed 3 times, and the supernatant fractions were pooled together. Purity of isolated fractions was quantitatively measured with flow cytometry (method described separately) and qualitatively evaluated with fluorescence microscopy (method described separately). Three independent trials were conducted to evaluate reproducibility. All conditions tested are summarized in Table 7.

TABLE 7
Conditions for the study of SMD specificity

| | Capture antibody type | MB type | DP |
|---|---|---|---|
| Condition 1: Blue bead | Goat anti-Rabbit/EP1 | CP1/CP2/CP3 | DP2 |
| (Mouse IgG coating) | Goat anti-Mouse/EP2 | | |
| release | Goat anti-Human/EP3 | | |
| Condition 2: Green bead | Goat anti-Rabbit/EP1 | CP1/CP2/CP3 | DP1 |
| (Rabbit IgG coating) | Goat anti-Mouse/EP2 | | |
| release | Goat anti-Human/EP3 | | |
| Condition 3: Red bead | Goat anti-Rabbit/EP1 | CP1/CP2/CP3 | DP3 |
| (Human IgG coating) | Goat anti-Mouse/EP2 | | |
| release | Goat anti-Human/EP3 | | |
| Condition 4: Control | Goat anti-Rabbit/EP1 | CP1/CP2/CP3 | None |
| (non-specific target | Goat anti-Mouse/EP2 | | |
| release) | Goat anti-Human/EP3 | | |

4-Color Bead Sorting with SMD Technology

Three sets of “target” fluorescent beads were prepared for this experiment: green beads were coated with rabbit IgG, blue beads with mouse IgG, and red beads with human IgG. Uncoated yellow beads were used as an “impurity” fraction. The four populations were pooled in even proportions into a single centrifuge tube to a total final concentration of 4×10⁶ beads/mL. DNA-antibody conjugates (Ab-EP) against rabbit, mouse, and human IgG were added to the mixture at a final concentration of 2.5 μg/mL each and incubated for 30 minutes. Meanwhile, 3 different CPs (CP1, CP2, and CP3) were mixed at a 1:2:2 ratio (5 μM total) and incubated with 3×10⁷ MBs for 30 minutes. Fluorescent beads were washed through centrifugation (at 3000 g for 2 minutes) 4 times to remove excess antibody, and MBs were washed 4 times with a magnet to remove excess CPs. The fluorescent beads were then mixed with MB-CP at a ratio of 50 MBs per target fluorescent bead and incubated for 30 minutes. The sample was then separated on a magnet for enrichment and isolation of green, blue, and red beads from yellow bead “impurity”. The sample was washed 3 times, and the supernatant fractions were pooled together. Following magnetic capture, DP3 (corresponding to red beads) was added to the sample at 5 μM final concentration, mixed gently with pipette for 1 minute, and then placed immediately on the magnet for collection of released beads in the supernatant. The sample was washed 3 times, and the supernatant fractions were pooled together. In the same way, displacement probes corresponding to blue and green beads (DP2 and DP1 respectively) were added sequentially, mixtures were washed 3 times, and supernatants were pooled. The remaining MB-bound fraction was also resuspended in equal volume of buffer to evaluate the amount of beads that were not released. PBS with 0.5% BSA was used throughout all steps of this study. All incubation steps were performed under gentle rotation at room temperature unless noted otherwise. Purity of isolated fractions was quantitatively measured with flow cytometry (method described separately) and qualitatively evaluated with fluorescence microscopy (method described separately). Three independent trials were conducted to evaluate reproducibility.

4-Color Bead Sorting with Sequential Streptavidin-Mediated Target Capture

Three sets of “target” fluorescent beads were prepared for this experiment: green beads were coated with rabbit IgG, blue beads with mouse IgG, and red beads with human IgG. Uncoated yellow beads were used as an “impurity” fraction. The four populations were pooled in even proportions into a single centrifuge tube to a total final concentration of 4×10⁶ beads/mL. First, for red bead capture, biotinylated goat anti-human antibody was added to the sample at 0.2 μg/mL, incubated for 15 minutes, and washed 4 times with centrifugation (at 3000 g for 2 minutes). The sample was then incubated with streptavidin-coated MBs (at 50 MBs per target bead ratio) for 30 minutes. The magnetic fraction was collected with the magnet to isolate captured “target” beads and washed 3 times. The supernatants with unbound beads were pooled together, pelleted through centrifugation (at 3000 g for 2 minutes), and resuspended back to the original sample volume of 100 μL for subsequent magnetic capture steps. In the same way, capture of blue and green “target” beads was performed through serial incubation with biotinylated secondary antibody (goat anti-mouse and goat anti-rabbit respectively), washing, incubation with MB-streptavidin, and magnetic collection. PBS with 0.5% BSA was used throughout all steps of this study. All incubation steps were performed under gentle rotation at room temperature. Purity of isolated fractions was quantitatively measured with flow cytometry (method described separately) and qualitatively evaluated with fluorescence microscopy (method described separately). Three independent trials were conducted to evaluate reproducibility.

### Study of SMD-Based Sorting Selectivity at Varying Target Abundance

Two sets of “target” fluorescent beads were prepared for this experiment: red beads were coated with human IgG, blue beads with mouse IgG. Samples were prepared at a blue: red bead ratio at 1:1, 1:5, 1:20, and 1:100 by maintaining the concentration of red bead at 1×10⁶ beads/mL and reducing amount of blue beads accordingly. To evaluate the performance of SMD technology during target capture at decreasing target concentrations, sample purity achieved with capture via biotin-streptavidin bond formation was compared to sample purity achieved with capture via oligonucleotide hybridization. For each blue: red bead ratio, the fluorescent beads were incubated with either biotinylated antibodies (goat anti-mouse, 0.2 μg/mL) or DNA-antibody conjugates (goat anti-mouse conjugated to EP2, 2.0 μg/mL) for 15 minutes and washed 4 times with centrifugation (at 3000 g for 2 minutes). Meanwhile, CP2-coated MBs were prepared by mixing 1×10⁷ MBs with CP2 at final DNA concentration of 1 μM, incubating for 15 minutes, and washing 4 times with magnet to remove excess CPs. Next, MB-Streptavidin (for samples previously incubated with biotinylated antibodies) or MB-CP2 (for samples previously incubated with DNA-antibody conjugates) were added to the sample at a constant concentration of 50 MBs per red fluorescent bead, incubated for 30 minutes, and isolated with using the magnet. Each magnetic fraction was washed 3 times, and the purity of the blue beads (rare target) in magnetic fraction was quantitatively measured with flow cytometry (method described separately).

To evaluate the performance of SMD during target release, both red and blue target beads were initially captured by MBs and then rare blue target beads were released via SMD. For each blue: red bead ratio, DNA-antibody conjugates against red (goat anti-human Ab/EP3) and blue (goat anti-mouse Ab/EP2) targets were added to the mixture at a final concentration of 2 μg/mL each, incubated for 15 minutes, and washed through centrifugation (at 3000 g for 2 minutes) 4 times. Meanwhile, complementary CPs (CP2 and CP3) were mixed at even proportion (l μM each) with 2×10⁷ MBs, incubated for 15 minutes, and washed 4 times with a magnet. The fluorescent beads were then mixed with MB-CP at a constant ratio of 50 MBs per red fluorescent bead, incubated for 30 minutes, isolated with a magnet (thus enriching both red and blue targets), and washed 3 times. Following magnetic capture, DP2 (corresponding to release of blue beads) was added to the sample at 5 μM final concentration, mixed gently with pipette for 1 minute, and placed immediately on the magnet for collection of released beads in the supernatant. The sample was washed 3 times, and the supernatant fractions were pooled together. The purity of blue beads (rare target) in the supernatant fraction was quantitatively measured with flow cytometry (method described separately). Three independent trials for each experiment were conducted to evaluate reproducibility. All conditions tested are summarized in Table 8.

TABLE 8
Conditions for the study of SMD selectivity at varying target abundance

| | Capture | | |
|---|---|---|---|
| | antibody type | MB type | DP |
| Condition 1: Blue bead | Goat anti- | Streptavidin | N/A |
| (Mouse IgG coating) capture | Mouse/Biotin | | |
| via biotin-streptavidin | | | |
| Condition 2: Blue bead | Goat anti-Mouse/ | CP2 | N/A |
| (Mouse IgG coating) capture | EP2 | | |
| via DNA-antibody conjugate | | | |
| Condition 3: Blue bead | Goat anti-Mouse/ | CP2/CP3 | DP2 |
| (Mouse IgG coating) release | EP2 | | |
| via SMD | Goat anti- | | |
| | Human/EP3 | | |

Quantitative Analysis with Flow Cytometry

Flow cytometry on LSR-II (BD Biosciences) machine was used to count the number of fluorescent beads for calculations of purity and yield of isolated fractions. In order to compare relative bead counts, all samples were reconstituted in the same 100 μL volume, and a 96-well plate setup was used to consistently analyze an equal volume of each sample. A total of 5 channels were used for bead identification and enumeration. Forward scatter (FSC-A) was used to discriminate particles based on size, such that small particulates were not included in further analysis. Four different excitation lasers and 4 band-pass filters were used to uniquely identify a single bead color, as listed in Table 9.

TABLE 9
Flow-cytometry parameters used for bead counting

| Microsphere | Laser | Emission filter |
|---|---|---|
| Blue | 350 nm | 450/40 nm |
| Green | 488 nm | 530/30 nm |
| Yellow | 561 nm | 610/20 nm |
| Red | 641 nm | 710/50 nm |

With this setup, beads were easily distinguished by their respective channel, and no compensation was necessary. For each specimen, at least 3000 beads were counted (lower counts for some cases where low target concentration was used). Flow cytometry data was analyzed in FlowJo 9.3.3 (TreeStar). The total number of fluorescent beads was calculated by summing the counts from each of the four excitation/emission channels. Purities are reported as the number of beads of one color, divided by the total number of fluorescent beads within the sample. The overall yield is reported as the number of beads isolated into their respective fraction, divided by the number of beads of the same color counted within the reference sample that did not undergo magnetic sorting.

Qualitative Analysis with Fluorescence Imaging

IX-71 inverted fluorescence microscope (Olympus) equipped with true-color camera (QColor5, Olympus) and spectral imaging camera (Nuance, CRI, covering 420-720 nm spectral range) was used for imaging of fluorescent beads. Low-magnification images were obtained with 20× dry objective (NA 0.75, Olympus) and high-magnification with either 40× oil-immersion objective (NA 1.30, Olympus) or 100× oil-immersion objective (NA 1.40, Olympus). Wide UV filter cube (330-385 nm band-pass excitation, 420 nm long-pass emission, Olympus) was used for imaging of blue beads, FITC LP cube (460-500 nm band-pass excitation, 510 nm long-pass emission, Chroma) for green beads, Rhodamine LP cube (530-560 nm band-pass excitation, 572 nm long-pass emission, Chroma) for yellow beads, and Cy5 LP cube (590-650 nm band-pass excitation, 665 nm long-pass emission, Chroma) for red beads. All images were acquired for beads deposited on the surface of a glass cover slip. For 4-color bead imaging, images obtained with individual filter cubes were false-colored and merged into a composite image in Photoshop (Adobe Systems). Background was removed, and brightness and contrast were adjusted for best visual representation and clarity.

### Results and Discussion

### Antigen-Coated Fluorescent Microspheres: a Model System for Cell Sorting

Although SMD-based magnetic sorting represents a platform technology applicable to a wide range of analytes, proof-of-concept work reported here most closely resembles conditions necessary for sorting of live cells, a significant application of this technology. In this setup, four-color fluorescent beads of size similar to mammalian cells are used as a model system for development and characterization of SMD technology. Fluorescent beads are easy to identify and count, and are thus ideal for technology characterization and validation. To magnetically isolate specific cell populations, antibodies against cell-specific surface markers are used. Thus, using fluorescent beads as a model system for cell sorting first requires surface modification with an identifying antigen. In these experiments, mouse, rabbit, and human IgGs were selected as the identifying antigens for blue, green, and red beads respectively; yellow beads were left unmodified to serve as an impurity population to remove. To attach antigen to the surface of carboxylated fluorescent beads, a two-step carbodiimide covalent conjugation protocol was developed (see Methods). To validate the conjugation procedure, the presence and density of a specific antigen on the surface of fluorescent beads, 2-step staining using biotinylated secondary antibodies for antigen detection and red-emitting quantum dots for fluorescent labeling was applied. Red quantum dots (emitting at 655 nm) exhibit very large Stokes shift and high brightness, enabling easy unmixing of Qdot signal from the fluorescence of target beads and quantitative analysis of staining intensity, while using single UV source for excitation of all samples. Blue, green, and red beads develop bright positive staining only for antigens that were conjugated to their surface (i.e. blue for mouse IgG, green for rabbit IgG, and red for human IgG), while showing no cross-reactivity with other antibodies or Qdots alone (control). Unconjugated yellow beads, which were used as an “impurity” population throughout all studies, failed to produce staining with any antibody, as expected.

### Kinetics and Specificity of Target Capture and Release

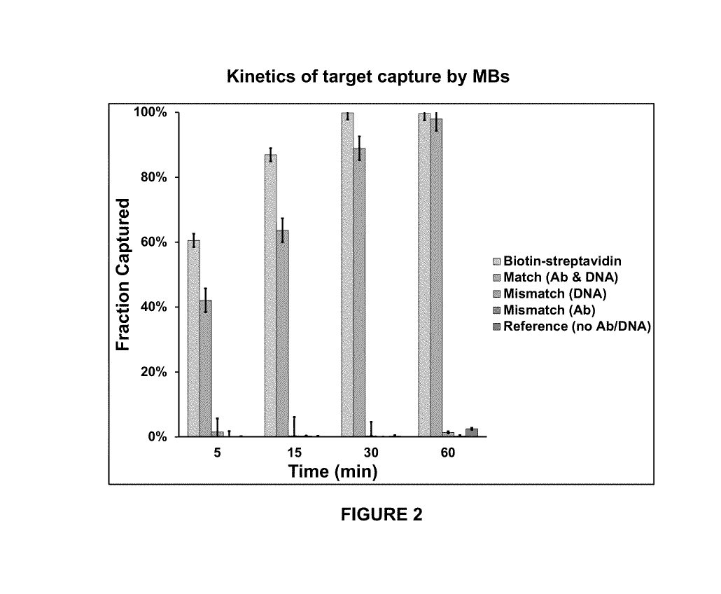

For any biomolecule separation method, both the overall performance and duration of the separation method are critical considerations. While maximizing the specificity of the method improves purity and yield, minimizing the duration of the separation procedure reduces the effort required for the procedure and helps preserve the biological characteristics of the target. Here, kinetics and specificity of the magnetic capture and SMD-based release were first evaluated using single-color green microspheres (see Methods, Table 2). As shown in FIG. 2, target capture through hybridization between EPs and CPs slightly lags behind that mediated by the gold standard streptavidin-biotin interaction, but both reach nearly complete target capture at a 1 hour time point (>98%). Fluorescence micrographs taken for the unbound (supernatant) and MB-bound fractions for matched and mismatched DNA sequences after a 60-min incubation period demonstrated that the matched EP1/CP1 pairs lead to nearly complete capture of target beads in the MB-bound fraction, whereas the mismatched sequences (CP2) produce only negligible non-specific binding (data not shown).

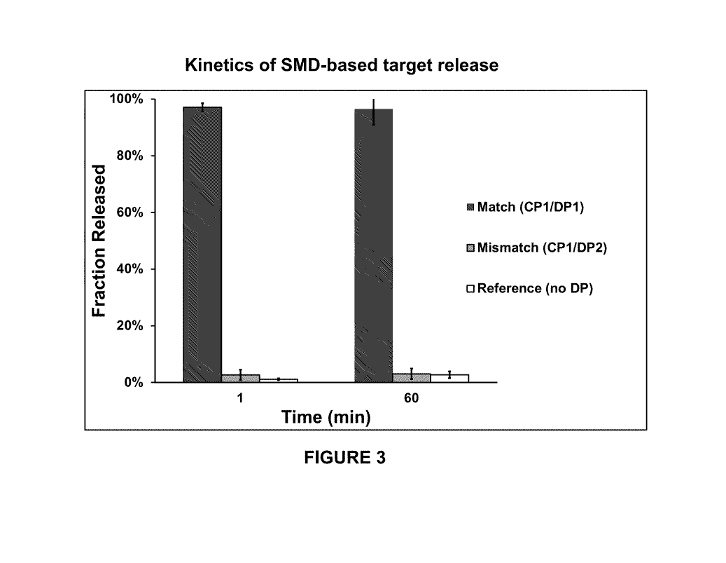

Following magnetic capture via DNA linkers, the specificity and kinetics of target release via SMD were evaluated (see Methods, Table 3). Target release was measured after 1 min or 60 min incubation periods (FIG. 3). Remarkably, even after 1 min of SMD, release of target beads into the supernatant is nearly complete (97%) for complementary DP, while only minimal nonspecific release (<3%) is observed for the mismatched DP and reference, demonstrating high selectivity and speed of SMD despite potential issues such as steric hindrance. Fluorescence microscopy (data not shown) further corroborated this conclusion. The outstanding kinetics and selectivity of SMD is enabled by three fundamental features of this technology. First, as mentioned earlier, DNA displacement with longer complementary strands is an extremely selective and fast process. Second, diffusion of small DNA strands to microbeads is much faster than diffusion between two microparticles, meaning the kinetics of target release can be much faster than target capture. Third, a profound impact originates from the differential concentrations in the separation reaction. In the case of conventional immunomagnetic separation, the two ‘reactants’ are MBs and targets, which are often used in fM to pM concentration range [18, 24]. In SMD, on the other hand, the two ‘reactants’ are MB-target complexes and single-stranded DNA with concentration typically in the μM range, which is six to nine orders of magnitude higher than that of MBs, thus driving the reaction rate. Noteworthy, there is virtually no non-specific target release for 1 min and 60 min incubation periods, indicating that the 16 base pair EP-CP overlap offers sufficient long-term stability (under gentle rotating agitation at room temperature). Yet, vigorous washing results in more noticeable non-specific target release with DNA links compared to biotin-streptavidin bond (data not shown). Such behavior is not surprising, as DNA-DNA binding strength (20-50 pN for 10-30 base pairs) [25] is considerably lower than that of biotin-streptavidin bond (300 pN) [26]. Poor bond stability can be partially addressed by using longer DNA probes, while maintaining binding specificity through careful sequence design. Furthermore, this effect may be negligible for separation of smaller analytes (proteins, bacteria), as shear forces decrease along with particle size.

The specificity of SMD technology was characterized in greater depth using four-color bead mixes, as the overall performance of the technology requires high specificity for each primary-secondary antibody pair and each set of DNA sequences. The specificity of target (blue, red, or green) capture from a 4-bead mix for standard magnetic isolation was tested (via streptavidin-biotin bond formation, see Methods, Table 4) (data not shown). The purity of magnetic fraction captured was calculated using flow cytometry as a measure of binding selectivity. For the standard capture method, specificity of antibody-antigen recognition was tested, while for SMD technology, both specificity of antibody-antigen recognition by DNA-antibody conjugates and specificity of DNA hybridization between encoding and capture probes were evaluated. Very high selectivity of target capture via EP-CP hybridization was obtained (with purities ranging from 96.9% to 98.9%). At the same time, antibodies exhibited some cross-reactivity, producing red bead impurity in green bead captured fraction (due to binding of anti-rabbit antibody to human IgG on red beads) and blue bead impurity in red bead captured fraction (due to binding of anti-human antibody to mouse IgG on blue beads) above background values in both streptavidin-biotin bond-mediated capture and SMD-based capture. In the control, only minor non-specific binding is observed between MBs and “target” (blue, green, and red) as well as “impurity” (yellow) beads.

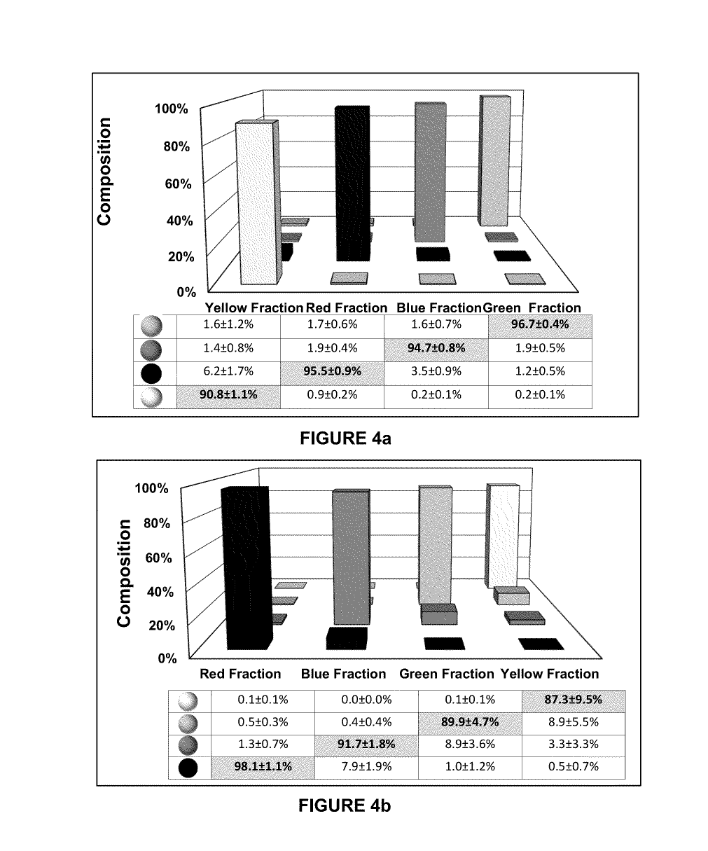

In a similar manner, specificity of SMD-based release of each target bead (blue, green, or red) from a MB-captured 3-bead mix was also tested to demonstrate the selectivity of each displacement probe sequence. The purity of isolated (unbound) fraction was calculated using flow cytometry as a measure of DNA displacement selectivity. Both qualitative evaluation with fluorescence microscopy and quantitative analysis of flow cytometry data indicate good selectivity of SMD, as evidenced by the moderate purities of isolated target fractions above ˜92%. Trace amounts of yellow beads are present in all fractions due to initial non-specific capture from a 4-bead mix. Some non-specific release of red and blue targets observed here might, in part, be explained by the lower antigen coating on these beads compared to green beads, as indicated by lower Qdot staining intensity discussed above. Effect of differential antigen surface coverage can be negated to some extent by adjusting the number of corresponding capture probes on MBs (i.e. increasing CP coverage on MB should improve binding with targets exhibiting lower density of surface antigen). Following this logic, SMD-based 4-bead sorting study presented in FIG. 4 (see next section) was performed using a 2:2:1 ratio of CPs on MBs for red, blue, and green targets respectively, which increased purity to ˜95% for each target. To further confirm that non-specific target release results from the rupture of DNA link due to mechanical forces rather than due to SMD by non-complementary DPs, we compared the fraction of beads released in cases with and without DP added (data not shown). As evident from the quantitative analysis of flow cytometry data, non-specific target release does not depend on the presence of DPs, showing no statistically significant difference between levels of impurities in all cases (p>0.05 based on two-tailed t-test), except for the blue bead impurity in the green bead fraction, which is actually lower than in control case.

### Characterization of SMD Technology Performance

We further characterize SMD performance in some magnetic separation scenarios, including (1) separation of a four-bead mixture into purified fractions using SMD technology, (2) a comparison with separation of a four-bead mixture using conventional sequential magnetic separation, and (3) the effects of varying target abundance on both separation procedures. First, the utility of SMD technology for quick sorting of multiple targets from a mixed sample was demonstrated (see Methods). In brief, four populations of fluorescent beads were first pooled in a single sample at even proportions. Beads of the three primary colors were tagged with distinct antigens on the surface (green with rabbit IgG, blue with mouse IgG, and red with human IgG), while unmodified yellow beads serve as an impurity for removal. Three antibodies specifically recognizing those surface antigens are tagged with unique encoding oligonucleotides (EP1, EP2, and EP3) and incubated with the mixture sample. In parallel, MBs are modified with CP1, CP2, and CP3, complementary to each EP. Following the procedure schematically illustrated in FIG. 1a, the red, blue and green beads are simultaneously enriched magnetically and subsequently isolated one at a time by addition of their cognate DPs. Qualitative evaluation with fluorescence microscopy (not shown) and quantitative analysis with flow cytometry revealed excellent purity (Red: 95.5%; Blue: 94.7%, and Green: 96.7%) and reproducibility (standard deviation <1%) for each of the isolated fractions (FIG. 4a). The overall yield for each target (Red: 68.2±13.8%, Blue: 74.5±8.5%, and Green: 61.4±8.2%) was calculated by dividing the number of beads collected by that in the reference sample, which did not undergo the magnetic separation procedure. The sources of the loss may include dead volume in pipette tips, retention of beads on centrifuge tube walls/caps, incomplete magnetic capture, nonspecific release, and incomplete release of targets. These parameters deserve future optimization especially for separation and characterization of rare targets, such as stem cells or circulating tumor cells. Nevertheless, the yield and throughput reported herein marks a considerable improvement over previous multi-target magnetic selection methods, while requiring no sophisticated instrumentation.

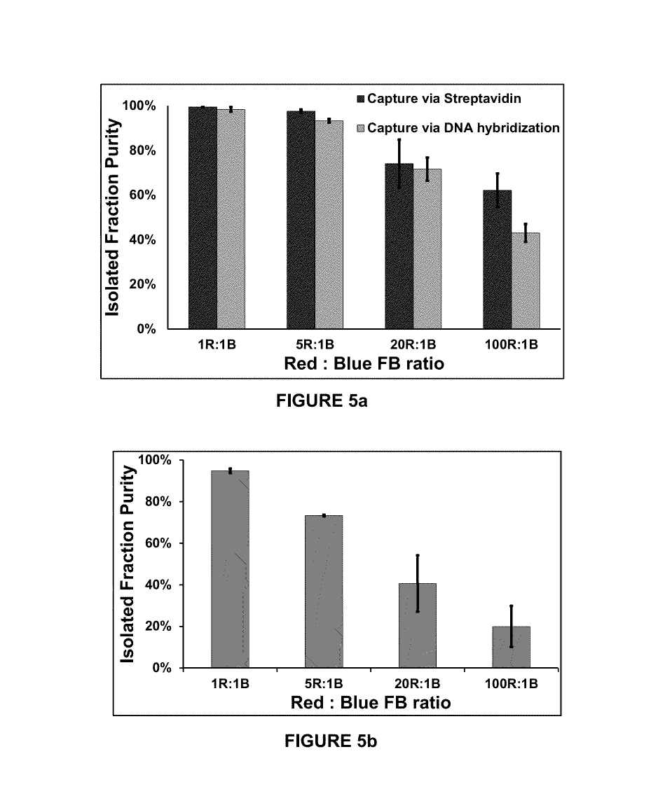

To further highlight the benefits of SMD technology, we contrasted our approach to conventional sequential immunomagnetic sorting (FIG. 4b). Here, using biotinylated antibodies and streptavidin-coated MBs, we again isolated 3 targets from an initial mixture of 4 bead populations (see Methods). From a 4-bead mixture (consisting of 3 “target” fluorescent beads and yellow “impurity” at equal proportions) targets were isolated in the following order: red beads->blue beads->green beads, leaving “impurity” yellow beads in unbound fraction. Both qualitative evaluation with fluorescence microscopy and quantitative analysis of flow cytometry data indicate high purity of the first isolated target (red, >98%) and markedly decreasing purity of subsequent isolation cycles (˜92% purity for blue and ˜90% for green beads), resulting mainly from contamination by the preceding target. Thus, not only is this process time consuming in comparison to SMD (requiring over 1.5 hours for each target isolated, for a total of ˜5 hours), but also incomplete magnetic capture of any given target creates impurities for targets isolated in downstream steps. We attributed this effect to incomplete magnetic capture of a given target, where biotinylated antibody remained on the target surface, creating a biotin-labeled impurity for capture via streptavidin during the next isolation cycle. Although this issue can potentially be circumvented by using unique secondary-affinity ligands, availability of such ligands is limited, falling far behind the multiplexing potential offered by DNA. At the same time, potential incomplete target release with SMD does not result in additional impurities in downstream fractions, as unique DPs are used for release of each target. Finally, we tested the dynamic range for multi-target SMD separation by systematically decreasing the ratio of target to impurity florescent beads, as large variations in target concentration can be expected with certain practical applications (FIG. 5). Expectedly, purity of target captured from a mix of beads in solution (FIG. 5a) as well as purity of target released from a MB-bound fraction containing multiple bead types (FIG. 5b) suffers from decreasing test target abundance (and increasing proportion of reference beads). With this, target capture demonstrates higher selectivity compared to target release (e.g. purity of capture via DNA hybridization drops from 98.4±1.0% to 43.0±4.0% with target abundance decreasing from 50% to ˜1%, whereas purity of release via SMD drops from 94.8±1.0% to 20.0±9.9%). However, it should be noted that an issue of isolation of rare targets at high purity is not unique to SMD technology, as similar drop in target purity at decreasing target abundance is observed with more conventional magnetic isolation via streptavidin-biotin bond formation (showing a drop of captured target purity from 99.5±0.2% at equal bead proportion to 62.1±7.5% at 1:100 test-to-reference bead ratio). Also, it is worth mentioning that the increase in error bar magnitude observed with decreasing test target abundance mainly results from a shot noise introduced by the low absolute number of test beads within the sample, with actual test bead counts from flow cytometry dropping from ˜3000 to only ˜30 when target abundance is decreased from 1:1 to 1:100. Inferring from the control studies and similar performance of biotin-streptavidin mediated capture (data not shown), we believe that major causes for this effect are antibody cross-reactivity, incomplete washing, and rupture of DNA links.

### CONCLUSIONS

In summary, the work described within this document demonstrates a simple yet robust multi-target immunomagnetic separation technology based on the concept of DNA strand-mediated displacement. Magnetic separation serves as a high-throughput platform for sorting of a wide array of targets, while DNA-antibody conjugates enable highly multiplexed indirect selection, which confers important benefits of high target yield and purity. Finally, rapid target sorting is enabled by SMD technology via fast DNA binding and displacement, fast diffusion of relatively small DPs, and high concentration of DNA reactants. Overall, combination of these critical components provides a unique solution to a long-standing problem in magnetic separation: multi-target sorting at high yield, purity, and throughput.

This work demonstrates that the versatility of SMD-based separation platform will enable a number of powerful applications, such as live cell sorting, as both target capture through DNA hybridization and SMD can be carried out in a range of cell- and bio-compatible buffers and at ambient or chilled conditions. Further, SMD technology can be used to allow further isolation via a different surface epitope of the same target, thus enabling multi-parameter selection. Finally, availability of DNA-antibody conjugates on target surface following MB release enables isolation of rare targets by applying several selective rounds of magnetic capture and SMD release.

SMD technology can be applied to the separation of any bioanalyate (i.e. cells, proteins, DNA), and thus applications for SMD technology extend beyond the cell-sorting application explored in-depth in these examples. For example, using SMD, it is possible to modify a traditional ELISA to capture and detect multiple antigens of interest by tagging each detection antibody with a specific DNA sequence. In one technique, incubation steps with complementary DNA-biotin, followed by binding with horseradish peroxidase (HRP)-streptavidin can be applied to complete the assay, making it possible to detect multiple antigens bound to a single well or MB sample through displacement and development of the DNA-biotin: streptavidin-HRP complex and color formation with a development buffer. In this setup, multiplexing is achieved via DNA-linkage rather than spatial multiplexing, thus circumventing the need to pre-spot plates.

### REFERENCES

1. Almand, B., et al., Clinical Significance of Defective Dendritic Cell Differentiation in Cancer. Clinical Cancer Research, 2000. 6(5): p. 1755-1766.
2. Mackensen, A., et al., Phase I Study of Adoptive T-Cell Therapy Using Antigen-Specific CD8+ T Cells for the Treatment of Patients With Metastatic Melanoma. Journal of Clinical Oncology, 2006. 24(31): p. 5060-5069.
3. Olsvik, O., et al., Magnetic separation techniques in diagnostic microbiology. Clin. Microbiol. Rev., 1994. 7(1): p. 43-54.
4. Stamm, C., et al., Autologous bone-marrow stem-cell transplantation for myocardial regeneration. The Lancet, 2003. 361(9351): p. 45-46.
5. Bailey, R. C., et al., DNA-Encoded Antibody Libraries: A Unified Platform for Multiplexed Cell Sorting and Detection of Genes and Proteins. Journal of the American Chemical Society, 2007. 129(7): p. 1959-1967.
6. Bonner, W. A., et al., Fluorescence Activated Cell Sorting. Review of Scientific Instruments, 1972. 43(3): p. 404-409.
7. Fu, A. Y., et al., A microfabricated fluorescence-activated cell sorter. Nature Biotechnology, 1999. 17(11): p. 1109-1111.
8. Miltenyi, S., et al., High gradient magnetic cell separation with MACS. Cytometry, 1990. 11(2): p. 231-238.
9. Use of magnetic techniques for the isolation of cells. Journal of Chromatography B: Biomedical Sciences and Applications, 1999. 722(1-2): p. 33-53.
10. Pruszak, J., et al., Markers and methods for cell sorting of human embryonic stem cell-derived neural cell populations. Stem Cells, 2007. 25(9): p. 2257-68.
11. Chalmers, J. J., et al., Flow Through, Immunomagnetic Cell Separation. Biotechnology Progress, 1998. 14(1): p. 141-148.
12. Inglis, D. W., et al., Continuous microfluidic immunomagnetic cell separation. Applied Physics Letters, 2004. 85(21): p. 5093-5095.
13. Liu, C., et al., On-chip separation of magnetic particles with different magnetophoretic mobilities. Journal of Applied Physics, 2007. 101(2): p. 024913.
14. Yavuz, C., et al., Low-Field Magnetic Separation of Monodisperse Fe3O4 Nanocrystals. Science, 2006. 314(5801): p. 964-967.
15. Yellen, B. B., et al., Traveling wave magnetophoresis for high resolution chip based separations. Lab on a Chip, 2007. 7(12): p. 1681-1688.
16. Adams, J. D., U. Kim, and H. T. Soh, Multitarget magnetic activated cell sorter. Proceedings of the National Academy of Sciences of the United States of America, 2008. 105(47): p. 18165-18170.
17. Semple, J. W., et al., Rapid separation of CD4+ and CD19+ lymphocyte populations from human peripheral blood by a magnetic activated cell sorter (MACS). Cytometry, 1993. 14(8): p. 955-60.
18. Tiwari, A., et al., Magnetic beads (Dynabead™) toxicity to endothelial cells at high bead concentration: Implication for tissue engineering of vascular prosthesis. Cell Biology and Toxicology, 2003. 19(5): p. 265-272.
19. Bath, J. and A. J. Turberfield, DNA nanomachines. Nature Nanotechnology, 2007. 2(5): p. 275-284.
20. Chen, Y. and C. Mao, Putting a Brake on an Autonomous DNA Nanomotor. Journal of the American Chemical Society, 2004. 126(28): p. 8626-8627.
21. Shin, J.-S. and N. A. Pierce, A Synthetic DNA Walker for Molecular Transport. Journal of the American Chemical Society, 2004. 126(35): p. 10834-10835.
22. Zhang, D. Y. and G. Seelig, Dynamic DNA nanotechnology using strand-displacement reactions. Nat Chem, 2011. 3(2): p. 103-13.
23. Yurke, B. and A. Mills, Using DNA to Power Nanostructures. Genetic Programming and Evolvable Machines, 2003. 4(2): p. 111-122.
24. Pilling, D., et al., The kinetics of interaction between lymphocytes and magnetic polymer particles. Journal of Immunological Methods, 1989. 122(2): p. 235-41.
25. Strunz, T., et al., Dynamic force spectroscopy of single DNA molecules. Proceedings of the National Academy of Sciences of the United States of America, 1999. 96(20): p. 11277-11282.
26. Zlatanova, J., S. M. Lindsay, and S. H. Leuba, Single molecule force spectroscopy in biology using the atomic force microscope. Progress in Biophysics and Molecular Biology, 2000. 74(1-2): p. 37-61.
27. Nie, C. Q., et al., CD4+ CD25+ Regulatory T Cells Suppress CD4+T-Cell Function and Inhibit the Development of Plasmodium berghei-Specific THJ Responses Involved in Cerebral Malaria Pathogenesis. Infection and Immunity, 2007. 75(5): p. 2275-2282.
28. Roberts, S. J., et al., Characterizing tumor-promoting T cells in chemically induced cutaneous carcinogenesis. Proceedings of the National Academy of Sciences of the United States of America, 2007. 104(16): p. 6770-6775.
29. Russell, M. S., et al., A Reduced Antigen Load In Vivo, Rather Than Weak Inflammation, Causes a Substantial Delay in CD8+T Cell Priming against Mycobacterium bovis (Bacillus Calmette-Guérin). Journal of Immunology, 2007. 179(1): p. 211-220.
30. Probst, C. E., P. Zrazhevskiy, and X. Gao, Rapid Multitarget Immunomagnetic Separation through Programmable DNA Linker Displacement. Journal of the American Chemical Society. 133(43): p. 17126-17129.

We claim:

1. A method for target isolation, comprising
(a) generating a first complex in a sample, wherein the first complex comprises:
(i) a first single stranded nucleic acid (NA1);
(ii) a second single stranded nucleic acid (NA2) bound to a surface, wherein the NA2 is complementary to the NA1, wherein the NA2 is bound to the NA1 via base pairing to form an NA1-NA2 complex comprising a double stranded region of at least 7 base pairs;
(iii) a first binding molecule (B1) bound to the NA1; and
(iv) a first target (T1) from the sample bound to the binding molecule; wherein the first complex comprises a T1-B1-NA1-NA2 complex;
(b) contacting the T1-B1-NA1-NA2 complex with a third single stranded nucleic acid (NA3); wherein the NA3 is complementary to a contiguous portion of either the NA1 or NA2 present in the double stranded region, such that hybridization of the NA3 to either the NA1 or NA2 is thermodynamically more favorable than the NA1-NA2 double stranded region; wherein the contacting occurs for a time and under conditions suitable to promote base pairing between the NA3 and either the NA1 and the NA2 to form a double stranded NA1-NA3 or NA2-NA3 region, whereby the NA1-NA2 double stranded region is disrupted; and
(c) isolating the T1 from the sample.

2. The method of claim 1, wherein generating the T1-B1-NA1-NA2 complex comprises:
(A) contacting the sample of interest with the NA1 bound to the B1; wherein the contacting occurs for a time and under conditions suitable for binding of the B1 to the T1 in the sample to form a T1-B1-NA1 complex;
(B) contacting the NA2 with the surface for a time and under conditions suitable to promote NA2 binding to the surface, thereby forming a surface-bound NA2;
(C) contacting the T1-B1-NA1 complex with the surface-bound NA2; wherein the contacting occurs for a time and under conditions suitable to promote formation of the NA1-NA2 complex, thereby forming the T1-B1-NA1-NA2 complex.

3. The method of claim 1, wherein generating the T1-B1-NA1-NA2 complex comprises:
(A) contacting the NA2 with the surface for a time and under conditions suitable to promote NA2 binding to the surface, thereby forming a surface-bound NA2;
(B) contacting the NA1 bound to the B1 with the surface-bound NA2; wherein the contacting occurs for a time and under conditions suitable to promote formation of the NA1-NA2 complex, thereby forming a B1-NA1-NA2 complex; and
(C) contacting the B1-NA1-NA2 complex with the sample of interest, wherein the contacting occurs for a time and under conditions suitable for binding of the B1 to the T1 in the sample to form the T1-B1-NA1-NA2 complex

4. The method of claim 1, wherein generating the T1-B1-NA1-NA2 complex comprises:
(A) contacting the sample of interest with the NA1 bound to the B1; wherein the contacting occurs for a time and under conditions suitable for binding of the B1 to the T1 in the sample to form a T1-B1-NA1 complex;
(B) contacting the T1-B1-NA1 complex with the NA2; wherein the contacting occurs for a time and under conditions suitable to promote formation of the NA1-NA2 complex; and
(C) contacting the T1-B1-NA1-NA2 complex with the surface for a time and under conditions suitable to promote NA2 binding to the surface, thereby forming the complex.

5. The method of claim 1, wherein steps (a) and (b) are each carried out two or more times.

6. The method of claim 1, wherein the NA1 is covalently bound to the binding molecule

7. The method of claim 1, wherein the surface comprises a surface selected from the group consisting of magnetic particles, microarrays, beads, columns, optical fibers, wipes, nitrocellulose, nylon, glass, quartz, diazotized membranes, silicones, polyformaldehyde, cellulose, cellulose acetate, paper, ceramics, metals, metalloids, semiconductive materials, coated beads, plastics, gel-forming materials, sol gels, porous polymer hydrogels, nanostructured surfaces, nanotubes, and nanoparticles.

8. The method of claim 1, wherein the NA1-NA2 complex double stranded region is between about 7 base pairs and about 30 base pairs in length.

9. The method of claim 1, wherein the NA1-NA2 complex double stranded region has a melting temperature of about 10° C. or above.

10. The method of claim 1, wherein the double stranded NA1-NA3 or NA2-NA3 region is between about 8 base pairs and about 50 base pairs in length.

11. The method of claim 1, wherein the B1 comprises an antibody specific for the target.

12. The method of claim 1, wherein the T1 is selected from the group consisting of cells, viruses, bacteria, proteins, polypeptides, nucleic acids, lipids, and carbohydrates.

13. The method of claim 1, further comprising
(d) generating a second complex in the sample, wherein the second complex comprises:
(i) a first single stranded nucleic acid (NA4);
(ii) a second single stranded nucleic acid (NA5) bound to a surface, wherein the NA5 is complementary to the NA4, wherein the NA5 is bound to the NA4 via base pairing to form an NA4-NA5 complex comprising a double stranded region of at least 7 base pairs;
(iii) a second binding molecule (B2) bound to the NA4; and
(iv) a second target (T2) from a sample of interest bound to the binding molecule; wherein the complex comprises a T2-B2-NA4-NA5 complex;
(e) contacting the T2-B2-NA4-NA5 complex with a third single stranded nucleic acid (NA6); wherein the NA6 is complementary to a contiguous portion of either the NA4 or NA5 present in the double stranded region, such that hybridization of the NA6 to either the NA4 or NA5 is thermodynamically more favorable than the NA4-NA5 double stranded region; wherein the contacting occurs for a time and under conditions suitable to promote base pairing between the NA6 and either the NA4 or the NA5, whereby the NA4-NA5 double stranded region is disrupted; and
(f) isolating the T2 from the sample.

14. The method of claim 13, wherein steps (b-c) and (e-f) are carried out sequentially following completion of steps (a) and (d).

15. A method for multiplexed target isolation, comprising
(a) generating a plurality of unique complexes in a sample, wherein each unique complex comprises:
(i) a unique first single stranded nucleic acid (UNA1);
(ii) a unique second single stranded nucleic acid (UNA2) bound to a surface, wherein the UNA2 is complementary to the UNA1, wherein the UNA2 is bound to the UNA1 via base pairing to form an UNA1-UNA2 complex comprising a double stranded region of at least 7 base pairs;
(iii) a unique binding molecule (UB) bound to the UNA1; and
(iv) a unique target (UT) from a sample of interest bound to the UB; wherein each unique complex comprises a UT-UB-UNA1-UNA2 complex;
(b) sequentially contacting each UT-UB-UNA1-UNA2 complex with a unique third single stranded nucleic acid (UNA3); wherein the UNA3 is complementary to a contiguous portion of either the UNA1 or UNA2 present in the double stranded region such that hybridization of the UNA3 to either the UNA1 or UNA2 is thermodynamically more favorable than the UNA1-UNA2 double stranded region; wherein the contacting occurs for a time and under conditions suitable to promote base pairing between the UNA3 and either the UNA1 or the UNA2, whereby the UNA1-UNA2 double stranded region is disrupted; and
(c) isolating each unique target from the sample.

16. The method of claim 15, further comprising enriching the plurality of unique complexes in the sample before carrying out step (b).

17. A kit, comprising:
(a) a first single stranded nucleic acid (NA1);
(b) a second single stranded nucleic acid (NA2) capable of binding to a surface, wherein the NA2 is complementary to the NA1, wherein the NA2 is capable of binding to the NA1 via base pairing to form an NA1-NA2 complex a double stranded region of at least 7 base pairs; and
(c) a third single stranded nucleic acid (NA3); wherein the NA3 is complementary to a contiguous portion of either the NA1 or NA2 present in the double stranded region such that hybridization of the NA3 to either the NA1 or NA2 is thermodynamically more favorable than the NA1-NA2 double stranded region.

18. The kit of claim 17, further comprising a binding molecule bound to the NA1.

19. The kit of claim 17, wherein the surface comprises a surface selected from the group consisting of magnetic particles, microarrays, beads, columns, optical fibers, wipes, nitrocellulose, nylon, glass, quartz, diazotized membranes, silicones, polyformaldehyde, cellulose, cellulose acetate, paper, ceramics, metals, metalloids, semiconductive materials, coated beads, plastics, gel-forming materials, sol gels, porous polymer hydrogels, nanostructured surfaces, nanotubes, and nanoparticles.
